SEQUENCE LISTING (96 sequences)

SEQ ID NO: 1 - protein, 8 aa, Artificial sequence
  HCDR1
GFTFTDYV

SEQ ID NO: 2 - protein, 8 aa, Artificial sequence
  HCDR2
IYPGGGFI

SEQ ID NO: 3 - protein, 8 aa, Artificial sequence
  HCDR3
FPKYYFDY

SEQ ID NO: 4 - protein, 11 aa, Artificial sequence
  LCDR1
QSIVHSNRYTY

SEQ ID NO: 5 - protein, 3 aa, Artificial sequence
  LCDR2
GVS

SEQ ID NO: 6 - protein, 9 aa, Artificial sequence
  LCDR3
FQSTLFPWT

SEQ ID NO: 7 - protein, 8 aa, Artificial sequence
  HCDR1
GYTFTEYT

SEQ ID NO: 8 - protein, 8 aa, Artificial sequence
  HCDR2
LNPNTGGT

SEQ ID NO: 9 - protein, 11 aa, Artificial sequence
  HCDR3
ARKGGWFFLDY

SEQ ID NO: 10 - protein, 5 aa, Artificial sequence
  LCDR1
SSVSY

SEQ ID NO: 11 - protein, 3 aa, Artificial sequence
  LCDR2
DTS

SEQ ID NO: 12 - protein, 9 aa, Artificial sequence
  LCDR3
QQWSSNPPT

SEQ ID NO: 13 - protein, 8 aa, Artificial sequence
  HCDR1
GFTFSSFG

SEQ ID NO: 14 - protein, 8 aa, Artificial sequence
  HCDR2
INSGDNYS

SEQ ID NO: 15 - protein, 16 aa, Artificial sequence
  HCDR3
ARPHIFYYGSPAWFAY

SEQ ID NO: 16 - protein, 11 aa, Artificial sequence
  LCDR1
QSLVHSNGNTY

SEQ ID NO: 17 - protein, 11 aa, Artificial sequence
  LCDR1
QSLVHSNKNTY

SEQ ID NO: 18 - protein, 3 aa, Artificial sequence
  LCDR2
KVS

SEQ ID NO: 19 - protein, 9 aa, Artificial sequence
  LCDR3
SQSTHVPLT

SEQ ID NO: 20 - protein, 8 aa, Artificial sequence
  HCDR1
GFTFSDYG

SEQ ID NO: 21 - protein, 8 aa, Artificial sequence
  HCDR2
ISSGSNTI

SEQ ID NO: 22 - protein, 14 aa, Artificial sequence
  HCDR3
ATMVTRWFYYAMDY

SEQ ID NO: 23 - protein, 5 aa, Artificial sequence
  LCDR1
SSVSY

SEQ ID NO: 24 - protein, 3 aa, Artificial sequence
  LCDR2
DTS

SEQ ID NO: 25 - protein, 9 aa, Artificial sequence
  LCDR3
QQWSSNPPT

SEQ ID NO: 26 - protein, 117 aa, Artificial sequence
  SHS007-17 VH
QVHLQQSGPELVKPGASVKMSCKASGFTFTDYVVSWVKQRTGQGLEWIGEIYPGGGFIHYNEKFKGKATLTADKSSNTAYMQLTSLTSEDSAVYFCARFPKYYFDYWGQGTTLTVSS

SEQ ID NO: 27 - protein, 112 aa, Artificial sequence
  SHS007-17 VL
DVVMTQTPLSLPVSLGDQASISCRSSQSIVHSNRYTYLEWYLQKPGQSLKLLIYGVSNRFSGVPDRFSGSGSGTDFTLKISRVEAEDMGVYYCFQSTLFPWTFGGGTKLEIK

SEQ ID NO: 28 - protein, 118 aa, Artificial sequence
  SHS007-39 VH
EVQLQQSGPELVKPGASVKISCKTSGYTFTEYTIHWMKQSHGKSLEWIGGLNPNTGGTTYNQKFKDKATLTVDKSSSTAYMALRSLTSEDSAVYYCARKGGWFFLDYWGQGTTLIVSS

SEQ ID NO: 29 - protein, 106 aa, Artificial sequence
  SHS007-39 VL
QIVLTQSPAIMSASPREKVTMTCSASSSVSYMHWYQQKSGTSPKRWIYDTSKLASGVPARFRGSGSGTSYSLTISNMEAEDAATYYCQQWSSNPPTFGGGTKVEIK

SEQ ID NO: 30 - protein, 123 aa, Artificial sequence
  SHS007-51 VH
EVQLVESGGDSVKPGGSLKLSCAASGFTFSSFGMSWVRQTPDKRLEWVATINSGDNYSYSPDSVKGRFTISRDNAKNILYLQMSSLKSEDTAMYYCARPHIFYYGSPAWFAYWGQGTLVTVSA

SEQ ID NO: 31 - protein, 112 aa, Artificial sequence
  SHS007-51 VL
DVVMTQTPLSLPVSLGDQASVSCRSRQSLVHSNGNTYLHWYLQRPGQSPKLLIYKVSNRFSGVPDRFSGSGSGTDFTLKISRVEAEDLGVYFCSQSTHVPLTFGAGTKLELK

SEQ ID NO: 32 - protein, 112 aa, Artificial sequence
  SHS007-51-G34K-CHI VL
DVVMTQTPLSLPVSLGDQASVSCRSRQSLVHSNKNTYLHWYLQRPGQSPKLLIYKVSNRFSGVPDRFSGSGSGTDFTLKISRVEAEDLGVYFCSQSTHVPLTFGAGTKLELK

SEQ ID NO: 33 - protein, 115 aa, Artificial sequence
  SHS007-59 VH
EVQLQQSGPELVKPGTSVKIPCKASGYTFTDYHMDWVKQSHGKSLEWIGDINPNNGGTIYNQKFKGKATLTVDKSSSTAYMELRGLTSEDTAVYYCARGGLHDYWGPGTTLTVSS

SEQ ID NO: 34 - protein, 107 aa, Artificial sequence
  SHS007-59 VL
DIVMTQSQKFMSTKVGDRVSITCKASQSVGTAVAWYQQKPGQSPKLLIYSTSNRYTGVPDRFTNNASGTDFTLTISNVQSADLADYFCQQYGNYPLTFGGGTKLEIK

SEQ ID NO: 35 - protein, 121 aa, Artificial sequence
  SHS007-65 VH
EVQLVESGGGLVKPGGSLKLSCAASGFTFSDYGIHWVRQAPEKGLEWVAYISSGSNTIYYADTVKGRFTISRDNAKNTLFLQMTSLRSEDTAIYYCATMVTRWFYYAMDYWGQGTSVTVSS

SEQ ID NO: 36 - protein, 106 aa, Artificial sequence
  SHS007-65 VL
QIVLTQSPAIMSASPGEKVTMTCSASSSVSYMYWYQQKSGTSPKRWIYDTSKLASGVPARFSGSGSGTSYSLTINNMEAEDAATYYCQQWSSNPPTFGAGTKLELK

SEQ ID NO: 37 - protein, 121 aa, Artificial sequence
  SHS007-72 VH
QVQLVQSGPELKKPGETVKISCKASGYTFTNYGMYWVKQAPGEGLKWMGWINTHTGEPTYGDDFKGRFAFSLETPATIAFLEISILKNEDTATYFCARKSFGDFIYAMDFWGQGTSVTVSS

SEQ ID NO: 38 - protein, 107 aa, Artificial sequence
  SHS007-72 VL
DIQMTQTTSSLSASLGDRVTISCRASQDIGKYLNWYQQKPDGTVKLLIYYTSRLHLGVPSRFSGSGSGTDFSLTISNLEQEDTATYFCQQGTTLPPTFGGGTKVEIK

SEQ ID NO: 39 - protein, 123 aa, Artificial sequence
  SHS007-90 VH
EVQLQQSGPELVKPGAAVQMSCQASGYTFTNYVIHWIQQKPGQGLEWVGYINPYNDGVNYNEKFKGKATLTSDKSSNTTYMEFRSLTSEDSAVYFCARWGNSYGHYGFALDYWGQGTSVTVSS

SEQ ID NO: 40 - protein, 106 aa, Artificial sequence
  SHS007-90 VL
QIVLSQSPTILSASPGEKVTMTCRPTSSVNYIHWFQQKPGSSPKPWIYASSSLVSGVPTRFSGRGSGTSYSLTISRVEAEDAATYYCQQWSGNPLTFGAGTKLEVR

SEQ ID NO: 41 - protein, 121 aa, Artificial sequence
  SHS007-95 VH
EVKLVESGGELVKPGGSLKLSCAASGFTLSSYVMSWVRQTPEKRLEWVAAISRYGDPTNFPDNMRDRFTISRDNAKNTLSLQMSSLRSEDTALYFCARHHFDGTSWYFDVWGPGTTVTVSS

SEQ ID NO: 42 - protein, 108 aa, Artificial sequence
  SHS007-95 VL
EIVLTQSPALMAASPGERVTITCSVSSSISSIDLHWYQQKSGTFPKLWIYGTSNLASGVPVRFSGSGSGTSYSLTISTMEAEDAATYYCQQWSSFPLTFGSGTKLEIK

SEQ ID NO: 43 - protein, 119 aa, Artificial sequence
  SHS007-108 VH
EVQLVESGGGLVQPGRSLKLSCLASGFTFNNYGMNWIRQAPGKGLEWVASISSSSSYIHYGDTVKGRFTISRDNAKNTLYLQVTSLRSEDTALYYCTRGLSYVGWFAHWGRGTLVTVSS

SEQ ID NO: 44 - protein, 107 aa, Artificial sequence
  SHS007-108 VL
DIQMTQSPASLSASLEEIVTITCQSSQNIGNWLAWYQQKPGKSPQLLIYGATTLADGVPSRFSGSRSGTQYSLKISRLQVEDIGIYYCQQASSTSWTFGGGTKLELK

SEQ ID NO: 45 - protein, 117 aa, Artificial sequence
  Humanized SHS007-17-CHI VH
QVQLVQSGAEVKKPGSSVKVSCKASGFTFTDYVVSWVRQAPGQGLEWMGEIYPGGGFIHYNEKFKGRVTITADKSTSTAYMELSSLRSEDTAVYYCARFPKYYFDYWGQGTTVTVSS

SEQ ID NO: 46 - protein, 117 aa, Artificial sequence
  Humanized SHS007-17-CHI VH
QVQLVQSGAEVKKPGSSVKVSCKASGFTFTDYVVSWVRQAPGQGLEWMGEIYPGGGFIHYNEKFKGRVTITADKSTSTAYMELSSLRSEDTAVYFCARFPKYYFDYWGQGTTVTVSS

SEQ ID NO: 47 - protein, 117 aa, Artificial sequence
  Humanized SHS007-17-CHI VH
QVQLVQSGAEVKKPGSSVKVSCKASGFTFTDYVVSWVRQAPGQGLEWIGEIYPGGGFIHYNEKFKGRATITADKSTSTAYMELSSLRSEDTAVYFCARFPKYYFDYWGQGTTVTVSS

SEQ ID NO: 48 - protein, 117 aa, Artificial sequence
  Humanized SHS007-17-CHI VH
QVQLVQSGAEVKKPGSSVKMSCKASGFTFTDYVVSWVKQAPGQGLEWIGEIYPGGGFIHYNEKFKGRATLTADKSTSTAYMELSSLRSEDTAVYFCARFPKYYFDYWGQGTTLTVSS

SEQ ID NO: 49 - protein, 112 aa, Artificial sequence
  Humanized SHS007-17-CHI VL
DIVMTQSPLSLPVTPGEPASISCRSSQSIVHSNRYTYLEWYLQKPGQSPQLLIYGVSNRFSGVPDRFSGSGSGTDFTLKISRVEAEDVGVYYCFQSTLFPWTFGQGTKVEIK

SEQ ID NO: 50 - protein, 112 aa, Artificial sequence
  Humanized SHS007-17-CHI VL
DIVMTQSPLSLPVTPGEPASISCRSSQSIVHSNRYTYLEWYLQKPGQSLQLLIYGVSNRFSGVPDRFSGSGSGTDFTLKISRVEAEDVGVYYCFQSTLFPWTFGQGTKVEIK

SEQ ID NO: 51 - protein, 112 aa, Artificial sequence
  Humanized SHS007-17-CHI VL
DVVMTQSPLSLPVTPGEPASISCRSSQSIVHSNRYTYLEWYLQKPGQSLQLLIYGVSNRFSGVPDRFSGSGSGTDFTLKISRVEAEDVGVYYCFQSTLFPWTFGQGTKLEIK

SEQ ID NO: 52 - protein, 118 aa, Artificial sequence
  Humanized SHS007-39-CHI VH
QVQLVQSGAEVKKPGASVKVSCKASGYTFTEYTIHWVRQAPGQGLEWMGGLNPNTGGTTYNQKFKDRVTMTRDTSISTAYMELSRLRSDDTAVYYCARKGGWFFLDYWGQGTLVTVSS

SEQ ID NO: 53 - protein, 118 aa, Artificial sequence
  Humanized SHS007-39-CHI VH
QVQLVQSGAEVKKPGASVKVSCKTSGYTFTEYTIHWVRQAPGQGLEWMGGLNPNTGGTTYNQKFKDRVTMTRDTSISTAYMELSRLRSDDTAVYYCARKGGWFFLDYWGQGTLVTVSS

SEQ ID NO: 54 - protein, 118 aa, Artificial sequence
  Humanized SHS007-39-CHI VH
QVQLVQSGAEVKKPGASVKVSCKTSGYTFTEYTIHWMRQAPGQGLEWMGGLNPNTGGTTYNQKFKDRVTMTRDTSISTAYMELSRLRSDDTAVYYCARKGGWFFLDYWGQGTLVTVSS

SEQ ID NO: 55 - protein, 118 aa, Artificial sequence
  Humanized SHS007-39-CHI VH
QVQLVQSGAEVKKPGASVKVSCKTSGYTFTEYTIHWMRQAPGQGLEWIGGLNPNTGGTTYNQKFKDRATMTRDTSISTAYMELSRLRSDDTAVYYCARKGGWFFLDYWGQGTLVTVSS

SEQ ID NO: 56 - protein, 118 aa, Artificial sequence
  Humanized SHS007-39-CHI VH
QVQLVQSGAEVVKPGASVKVSCKTSGYTFTEYTIHWMRQAPGQGLEWIGGLNPNTGGTTYNQKFKDKATMTVDTSISTAYMELSRLRSDDTAVYYCARKGGWFFLDYWGQGTLVTVSS

SEQ ID NO: 57 - protein, 118 aa, Artificial sequence
  Humanized SHS007-39-CHI VH
QVQLVQSGAEVVKPGASVKISCKTSGYTFTEYTIHWMKQAPGQGLEWIGGLNPNTGGTTYNQKFKDKATLTVDTSISTAYMELSRLRSDDTAVYYCARKGGWFFLDYWGQGTLLTVSS

SEQ ID NO: 58 - protein, 106 aa, Artificial sequence
  Humanized SHS007-39-CHI VL
EIVLTQSPATLSLSPGERATLSCSASSSVSYMHWYQQKPGQAPRLLIYDTSKLASGIPARFSGSGSGTDFTLTISSLEPEDFAVYYCQQWSSNPPTFGGGTKVEIK

SEQ ID NO: 59 - protein, 106 aa, Artificial sequence
  Humanized SHS007-39-CHI VL
EIVLTQSPATLSLSPGERATLSCSASSSVSYMHWYQQKPGQAPRRLIYDTSKLASGIPARFSGSGSGTDFTLTISSLEPEDFAVYYCQQWSSNPPTFGGGTKVEIK

SEQ ID NO: 60 - protein, 106 aa, Artificial sequence
  Humanized SHS007-39-CHI VL
EIVLTQSPATLSLSPGERATLSCSASSSVSYMHWYQQKPGQAPRRLIYDTSKLASGIPARFSGSGSGTDYTLTISSLEPEDFAVYYCQQWSSNPPTFGGGTKVEIK

SEQ ID NO: 61 - protein, 106 aa, Artificial sequence
  Humanized SHS007-39-CHI VL
EIVLTQSPATLSLSPGERATLSCSASSSVSYMHWYQQKPGQAPRRLIYDTSKLASGVPARFSGSGSGTDYTLTISSLEPEDAATYYCQQWSSNPPTFGGGTKVEIK

SEQ ID NO: 62 - protein, 106 aa, Artificial sequence
  Humanized SHS007-39-CHI VL
EIVLTQSPATLSASPGERVTMSCSASSSVSYMHWYQQKPGQAPRRLIYDTSKLASGVPARFSGSGSGTDYTLTISSMEPEDAATYYCQQWSSNPPTFGGGTKVEIK

SEQ ID NO: 63 - protein, 123 aa, Artificial sequence
  Humanized SHS007-51-G34K-2 VH
EVQLVESGGGLVKPGGSLRLSCAASGFTFSSFGMSWVRQAPGKGLEWVSTINSGDNYSYSPDSVKGRFTISRDNAKNSLYLQMNSLRAEDTAVYYCARPHIFYYGSPAWFAYWGQGTLVTVSS

SEQ ID NO: 64 - protein, 123 aa, Artificial sequence
  Humanized SHS007-51-G34K-2 VH
EVQLVESGGGLVKPGGSLRLSCAASGFTFSSFGMSWVRQAPGKGLEWVATINSGDNYSYSPDSVKGRFTISRDNAKNSLYLQMNSLRAEDTAVYYCARPHIFYYGSPAWFAYWGQGTLVTVSS

SEQ ID NO: 65 - protein, 112 aa, Artificial sequence
  Humanized SHS007-51-G34K-2 VL
DVVMTQSPLSLPVTLGQPASISCRSRQSLVHSNKNTYLHWFQQRPGQSPRRLIYKVSNRFSGVPDRFSGSGSGTDFTLKISRVEAEDVGVYYCSQSTHVPLTFGGGTKVEIK

SEQ ID NO: 66 - protein, 112 aa, Artificial sequence
  Humanized SHS007-51-G34K-2 VL
DVVMTQSPLSLPVTLGQPASISCRSRQSLVHSNKNTYLHWYQQRPGQSPRRLIYKVSNRFSGVPDRFSGSGSGTDFTLKISRVEAEDVGVYYCSQSTHVPLTFGGGTKVEIK

SEQ ID NO: 67 - protein, 112 aa, Artificial sequence
  Humanized SHS007-51-G34K-2 VL
DVVMTQSPLSLPVTLGQPASISCRSRQSLVHSNKNTYLHWFQQRPGQSPRLLIYKVSNRFSGVPDRFSGSGSGTDFTLKISRVEAEDVGVYYCSQSTHVPLTFGGGTKVEIK

SEQ ID NO: 68 - protein, 112 aa, Artificial sequence
  Humanized SHS007-51-G34K-2 VL
DVVMTQSPLSLPVTLGQPASISCRSRQSLVHSNKNTYLHWYQQRPGQSPRLLIYKVSNRFSGVPDRFSGSGSGTDFTLKISRVEAEDVGVYYCSQSTHVPLTFGGGTKVEIK

SEQ ID NO: 69 - protein, 112 aa, Artificial sequence
  Humanized SHS007-51-G34K-2 VL
DVVMTQSPLSLPVTLGQPASISCRSRQSLVHSNKNTYLHWYQQRPGQSPRLLIYKVSNRFSGVPDRFSGSGSGTDFTLKISRVEAEDVGVYFCSQSTHVPLTFGGGTKVEIK

SEQ ID NO: 70 - protein, 112 aa, Artificial sequence
  Humanized SHS007-51-G34K-2 VL
DVVMTQSPLSLPVTLGQPASVSCRSRQSLVHSNKNTYLHWYQQRPGQSPRLLIYKVSNRFSGVPDRFSGSGSGTDFTLKISRVEAEDVGVYFCSQSTHVPLTFGGGTKLEIK

SEQ ID NO: 71 - protein, 121 aa, Artificial sequence
  Humanized SHS007-65-CHI VH
EVQLVESGGGLVQPGGSLRLSCAASGFTFSDYGIHWVRQAPGKGLEWVSYISSGSNTIYYADTVKGRFTISRDNAKNSLYLQMNSLRAEDTAVYYCARMVTRWFYYAMDYWGQGTTVTVSS

SEQ ID NO: 72 - protein, 121 aa, Artificial sequence
  Humanized SHS007-65-CHI VH
EVQLVESGGGLVQPGGSLRLSCAASGFTFSDYGIHWVRQAPGKGLEWVSYISSGSNTIYYADTVKGRFTISRDNAKNSLYLQMNSLRAEDTAVYYCATMVTRWFYYAMDYWGQGTTVTVSS

SEQ ID NO: 73 - protein, 121 aa, Artificial sequence
  Humanized SHS007-65-CHI VH
EVQLVESGGGLVQPGGSLRLSCAASGFTFSDYGIHWVRQAPGKGLEWVAYISSGSNTIYYADTVKGRFTISRDNAKNSLYLQMNSLRAEDTAVYYCATMVTRWFYYAMDYWGQGTTVTVSS

SEQ ID NO: 74 - protein, 106 aa, Artificial sequence
  Humanized SHS007-65-CHI VL
EIVLTQSPATLSLSPGERATLSCSASSSVSYMYWYQQKPGQAPRLLIYDTSKLASGIPARFSGSGSGTDFTLTISSLEPEDFAVYYCQQWSSNPPTFGQGTKLEIK

SEQ ID NO: 75 - protein, 106 aa, Artificial sequence
  Humanized SHS007-65-CHI VL
EIVLTQSPATLSLSPGERATLSCSASSSVSYMYWYQQKPGQAPRRLIYDTSKLASGIPARFSGSGSGTDFTLTISSLEPEDFAVYYCQQWSSNPPTFGQGTKLEIK

SEQ ID NO: 76 - protein, 106 aa, Artificial sequence
  Humanized SHS007-65-CHI VL
EIVLTQSPATLSLSPGERATLSCSASSSVSYMYWYQQKPGQAPRRLIYDTSKLASGIPARFSGSGSGTDYTLTISSLEPEDFAVYYCQQWSSNPPTFGQGTKLEIK

SEQ ID NO: 77 - protein, 106 aa, Artificial sequence
  Humanized SHS007-65-CHI VL
EIVLTQSPATLSLSPGERATLSCSASSSVSYMYWYQQKPGQAPRRLIYDTSKLASGVPARFSGSGSGTDYTLTISSLEPEDFAVYYCQQWSSNPPTFGQGTKLEIK

SEQ ID NO: 78 - protein, 106 aa, Artificial sequence
  Humanized SHS007-65-CHI VL
EIVLTQSPATLSASPGERVTLSCSASSSVSYMYWYQQKPGQAPRRLIYDTSKLASGVPARFSGSGSGTDYTLTISSLEPEDFAVYYCQQWSSNPPTFGQGTKLEIK

SEQ ID NO: 79 - protein, 106 aa, Artificial sequence
  Humanized SHS007-65-CHI VL
EIVLTQSPATLSASPGERVTMSCSASSSVSYMYWYQQKPGQAPRRLIYDTSKLASGVPARFSGSGSGTDYTLTISSMEPEDFAVYYCQQWSSNPPTFGQGTKLEIK

SEQ ID NO: 80 - DNA, 351 bp, Artificial sequence
  hu17-00 VH nucleotide sequence
caggtgcagctggttcagagcggcgctgaggtgaagaagcccggcagcagcgtgaaggtgagctgcaaggccagcggctttacctttaccgactacgtggtaagctgggtgaggcaggcaccgggccagggcctggagtggatgggcgagatctaccctggcggaggattcattcactacaacgagaagttcaagggcagggtgaccatcaccgctgacaagagcacaagcaccgcctacatggagctgtcaagcctgaggagcgaggacaccgccgtgtattactgcgccaggttccccaaatactatttcgactactggggccaaggcaccaccgtgaccgtgagcagc

SEQ ID NO: 81 - DNA, 336 bp, Artificial sequence
  hu17-00 VL nucleotide sequence
gacatcgtgatgacccagagccccctgagcctgcccgtgaccccaggcgagcccgccagcatcagctgcaggagcagccagagcatcgtgcacagcaacaggtacacctacctggagtggtacctgcagaagcccggtcagtcaccacagctgctgatctacggcgtgagcaaccgcttcagcggcgtgcccgacaggtttagcggaagcggcagcgggaccgacttcaccctgaagatctctagggtggaggccgaggacgtgggcgtgtactactgcttccagtctaccttgttcccctggaccttcggccagggcaccaaggtggagatcaag

SEQ ID NO: 82 - DNA, 354 bp, Artificial sequence
  hu39-01 VH nucleotide sequence
caggtgcagctggtgcagagtggggccgaggtgaaaaagcccggtgccagcgtgaaagtgagctgcaaggccagcggctataccttcaccgagtacaccatccattgggtgaggcaggcacccggccagggcctggagtggatgggcggtctgaaccccaacaccggcggaaccacctacaaccaaaagttcaaggaccgcgtaactatgaccagggacaccagcatcagcaccgcctacatggagctgagcaggctgaggagcgacgacaccgccgtgtactactgcgccaggaagggcggctggttcttcttggactactggggccaaggcaccttggtgactgttagctct

SEQ ID NO: 83 - DNA, 318 bp, Artificial sequence
  hu39-01 VL nucleotide sequence
gagatcgtgctgacccagagccccgctactctgagcctgagccctggcgagagggccacgctgagctgctctgcctcaagcagcgtgagctacatgcattggtatcagcagaagcccggacaggcccccaggcgcttgatctacgacaccagcaagctggcctctggcatccccgccaggttcagcggcagcggctccggcaccgacttcaccctgaccataagcagcctggagcccgaggacttcgccgtgtactactgccagcagtggagcagcaacccccccaccttcggcggaggcaccaaggtggagatcaag

SEQ ID NO: 84 - DNA, 369 bp, Artificial sequence
  hu51-G34K-12 VH nucleotide sequence
gaggtgcagctggtggagagcggtggcggtctggtgaaacccggtggcagcctgaggctgagttgtgcggcgagcggcttcaccttcagcagcttcggcatgagctgggtgaggcaagcccctggcaagggcttggagtgggtggctaccatcaacagcggcgacaactacagctacagccccgacagcgtgaagggcaggttcaccatcagcagggacaacgcgaagaactccctgtacctgcagatgaacagccttagggccgaagacaccgccgtgtactactgcgccaggccccacatcttctactacggctcccccgcatggttcgcctactggggccagggcaccctggtgactgtgagcagc

SEQ ID NO: 85 - DNA, 336 bp, Artificial sequence
  hu51-G34K-12 VL nucleotide sequence
gacgtggtgatgacccagagccccctcagcctgcccgtgaccctcggacagccggcaagcatcagctgcaggagcaggcagagcctggtgcacagcaacaagaacacctacctgcactggttccagcaaaggcctggccaatcacccaggctgctgatctacaaggtgagcaacaggttcagcggcgtgcccgacaggtttagtggcagtggctcaggcaccgattttaccctgaagattagcagggtggaggccgaggacgtgggcgtgtactactgcagccagagcacccatgtgcctctgaccttcggtggcggaactaaggtggagatcaag

SEQ ID NO: 86 - DNA, 363 bp, Artificial sequence
  hu65-13 VH nucleotide sequence
gaggtgcagctggtggagagcggaggcggcctggtacagcccggtggctcactgaggctgagctgtgccgccagcggcttcaccttcagcgactacggcatccactgggtgcgacaagcccctggcaagggcctggagtgggtgagctacatcagcagcggcagcaacaccatctactacgccgacacagtgaagggcaggttcaccatttctagggacaacgccaagaactctctttacctgcagatgaacagcctgagggccgaagacaccgccgtgtactactgcgccaccatggtgaccaggtggttctattacgctatggactactggggccagggcaccaccgtgactgtgagcagc

SEQ ID NO: 87 - DNA, 318 bp, Artificial sequence
  hu65-13 VL nucleotide sequence
gagatcgtgctgacccagagccccgcaacactgagcctgagtcccggcgagagggccactctgagctgctcagccagcagcagcgtgagctatatgtattggtatcagcagaagcctggccaagcccccaggcggttgatctacgacaccagcaagctggcaagcggcgtccccgccaggttcagtggtagcggcagcgggacagactacaccctgaccatctccagccttgagcccgaggacttcgccgtgtactactgccagcaatggagcagcaacccccccaccttcggacagggaaccaaactggaaatcaag

SEQ ID NO: 88 - protein, 506 aa, Artificial sequence
  4Ig-B7-H3 extracellular domain
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 89 - protein, 217 aa, Artificial sequence
  2Ig-B7-H3 extracellular domain
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFP

SEQ ID NO: 90 - DNA, 1602 bp, Artificial sequence
  4Ig-B7-H3 full-length cDNA
atgctgcggaggagaggaagccctggcatgggtgtccatgtgggggcagcacttggagccctgtggttttgcctcaccggggcactcgaggtccaggtgcctgaggatccggttgtggccctcgtagggacagacgcgactctttgctgctccttctctcccgagcctgggtttagccttgcccagctcaacctgatttggcagctgacggacaccaagcagttggtccactctttcgccgagggacaagaccagggcagcgcgtatgccaacagaaccgccctttttcctgatctcttggctcaaggcaacgctagtctgcggctgcaaagggtccgggtggctgacgaagggagtttcacctgctttgtgagcatccgagactttggtagcgcagccgttagtttgcaggtggcagccccctatagcaagccatccatgaccctggaacccaataaggatctgaggccgggtgacacggtgaccatcacgtgtagcagctatcagggctatccagaggctgaggttttctggcaggacggccaaggcgtgccgcttaccggaaacgtgaccacgagccaaatggcgaatgaacaagggctgtttgacgtacactctatcctgagagtggtgttgggcgctaacgggacctatagttgtttggtcagaaatcccgtccttcaacaagacgcgcatagcagcgtcacgatcacgccccaaaggagcccgacaggggccgtagaggttcaagtacccgaagacccagtcgtggcgttggttggaacagatgccaccctgaggtgtagctttagcccggaacccggcttctccctggcccaacttaacttgatctggcaattgaccgacactaagcagctggtgcacagcttcacagagggcagggatcaagggagcgcctacgcaaataggaccgcactgtttcccgatctgctcgcccaaggtaacgcgtccctgagactgcagcgggtgagggtcgccgacgagggatcttttacatgttttgttagtattcgcgacttcggttcagccgccgtgagcctgcaagttgcagcgccatattctaaaccttctatgacattggaaccaaataaagaccttcgcccaggcgacacagtaacaataacgtgttctagttacaggggatacccggaggcggaggtattttggcaagacggtcaaggtgtgcccctgaccggcaatgtaaccaccagccagatggctaatgagcaaggcctgttcgacgtgcactcagtattgcgggtcgtgcttggcgcgaatggcacgtattcttgtctggtgaggaatccagttctgcaacaggacgcccacggaagtgttaccataacagggcaaccgatgacttttccccccgaagcactctgggtaaccgtaggcctgagtgtgtgcctcatcgcgcttttggtggcactggcgttcgtgtgctggaggaagatcaaacaaagctgcgaagaggagaacgccggtgccgaggaccaagatggagaaggcgagggtagcaaaacggcactgcaacccctgaaacacagcgacagcaaggaagacgatggacaggagatagcc

SEQ ID NO: 91 - protein, 836 aa, Artificial sequence
  4Ig-B7-H3 Fc fusion protein
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 522 aa, Artificial sequence
  4Ig-B7-H3 His fusion protein
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIAEQKLISEEDLHHHHHH

SEQ ID NO: 93 - protein, 547 aa, Artificial sequence
  2Ig-B7-H3 Fc fusion protein
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 233 aa, Artificial sequence
  2Ig-B7-H3 His fusion protein
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPEQKLISEEDLHHHHHH

SEQ ID NO: 95 - protein, 452 aa, Artificial sequence
  hBRCA84D heavy chain
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 96 - protein, 214 aa, Artificial sequence
  hBRCA84D light chain
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0332829 A1
(43) Pub. Date: Oct. 20, 2022

(54) ANTI-B7-H3 ANTIBODY AND APPLICATION THEREOF

(71) Applicant: NANJING SANHOME PHARMACEUTICAL CO., LTD., Nanjing, Jiangsu (CN)

(72) Inventors: Yong WANG, Nanjing, Jiangsu (CN); Liwen ZHAO, Nanjing, Jiangsu (CN); Yang XIAO, Nanjing, Jiangsu (CN); Yao XU, Nanjing, Jiangsu (CN); Cheng LUO, Nanjing, Jiangsu (CN); Yang YANG, Nanjing, Jiangsu (CN); Aiqi WEI, Nanjing, Jiangsu (CN)

(73) Assignee: NANJING SANHOME PHARMACEUTICAL CO., LTD., Nanjing, Jiangsu (CN)

(21) Appl. No.: 17/760,738

(22) PCT Filed: Sep. 15, 2020
(86) PCT No.: PCT/CN2020/115262

§ 371 Date: Mar. 15, 2022

(30) Foreign Application Priority Data
Sep. 16, 2019 (CN) 201910872843.3

(51) Int. Cl. C07K 16/28; A61P 35/00

(52) U.S. Cl. CPC C07K 16/2827; A61P 35/00; A61K 2039/505

(57) ABSTRACT

Provided is an anti-B7-H3 antibody or antigen-binding fragment thereof, a pharmaceutical composition containing the anti-B7-H3 antibody or antigen-binding fragment thereof, and an application thereof. The anti-B7-H3 antibody or antigen-binding fragment thereof of the present invention has high affinity and stability, and can be used for preparing antitumor drugs.

### FIELD

The present disclosure relates to the technical field of antibody drugs, and in particular, to an anti-B7-H3 antibody or an antigen-binding fragment thereof, a pharmaceutical composition comprising the anti-B7-H3 antibody or the antigen-binding fragment thereof, and uses thereof.

### BACKGROUND

B7-H3 (CD276), a member of the B7 superfamily, is a transmembrane glycoprotein, and its extracellular domain has two types of structure, one is monovalent 2Ig-B7-H3, the other is bivalent 4Ig-B7-H3 composed of two repeating units. As a novel immune checkpoint, B7-H3 has been reported in the literature to inhibit T cell proliferation and cytokine release (Suh W K, et al. The B7 family member B7-H3 preferentially down-regulates T helper type 1-mediated immune responses[J]. Nature Immunology, 2003, 4(9):899) and inhibit the lytic activity of NK cells against patient-derived tumor cells with high B7-H3 expression (Castriconi R, et al. Identification of 4Ig-B7-H3 as a neuroblastoma-associated molecule that exerts a protective role from an NK cell-mediated lysis[J]. Proceedings of the National Academy of Sciences, 2004, 101(34):12640-12645) by interacting with a receptor with unknown structure. Although the B7-H3 receptor is unknown, an increasing number of reports have been published in recent years on the negative regulation by the interaction of B7-H3 with its receptor in tumor immunity. Tumor cells may express B7-H3 to circumvent the immune surveillance of CD8+ T cells. In mouse models, the use of anti-mouse B7-H3 antibodies can enhance the immune function of CD8+ T cells (Yonesaka K, et al. B7-H3 negatively modulates CTL-mediated cancer immunity [J]. Clinical Cancer Research, 2018, clincanres.2852.2017). In the study of Lee Y, et al., gene knockout of mouse B7-H3 or the use of anti-mouse B7-H3 antibody can significantly inhibit tumor growth, and this inhibition depends on the function of CD8+T and NK cells (Lee Y, et al. Inhibition of the B7-H3 immune checkpoint limits tumor growth by enhancing cytotoxic lymphocyte function[J]. Cell Research, 2017, 27(8):1034-1045). Studies in patients with lung cancer have found that patients with high B7-H3 expression have fewer tumor-infiltrating lymphocytes and are more susceptible to lymph node metastasis (Altan M, et al. B7-H3 expression in NSCLC and its association with B7-H4, PD-L1 and Tumor Infiltrating Lymphocytes[J]. Clinical Cancer Research, 2017, 23(17):5202).

B7-H3 is abnormally expressed in various tumor cells, tumor stroma, tumor blood vessels, and tumor-infiltrating macrophages and DC cells. In tissue samples from patients with prostate cancer, pancreatic cancer, hepatocellular carcinoma, head and neck cancer, kidney cancer, etc., the expression rate of B7-H3 exceeds 90%, while it is rarely expressed in normal tissues (Seaman S, et al. Eradication of Tumors through Simultaneous Ablation of CD276/B7-H3-Positive Tumor Cells and Tumor Vasculature.[J]. Cancer Cell, 2017, 31(4):501-515). Based on the tumor-specific expression trend of B7-H3, the antibody drugs against B7-H3 target under clinical research are all developed with the help of the tumor targeting of B7-H3. Enoblituzumab, a humanized anti-B7-H3 monoclonal antibody, kills tumors by targeting the tumor-mediated ADCC effect of the Fc region of the antibody. Burtomab is a murine-derived anti-B7-H3 antibody-drug conjugate carrying radioactive iodine, and is used in the clinical treatment of metastatic neuroblastoma, which significantly improves the survival of children with cancer.

Based on the high expression characteristics of B7-H3 in the tumor environment and its inhibitory effect on immune cells, B7-H3 may be an effective target for the development of anti-tumor drugs. It is necessary to provide an anti-B7-H3 antibody or an antigen-binding fragment thereof with higher affinity and stability, which can block the immunosuppression of B7-H3 on tumors, so that enhance the killing activity of CD8+ T cells and NK cells on tumors, and synergize with the ADCC effect of the Fc fragment of the antibody to play an anti-tumor effect, giving more excellent anti-tumor activity.

### SUMMARY

One aspect of the present disclosure provides an anti-B7-H3 antibody or antigen-binding fragment thereof, comprising a heavy chain variable region and/or a light chain variable region, wherein the heavy chain variable region comprises complementarity determining region 1 of the heavy chain variable region (HCDR1), complementarity determining region 2 of the heavy chain variable region (HCDR2), and/or complementarity determining region 3 of the heavy chain variable region (HCDR3); and the light chain variable region comprises complementarity determining region 1 of the light chain variable region (LCDR1), complementarity determining region 2 of the light chain variable region (LCDR2), and/or complementarity determining region 3 of the light chain variable region (LCDR3).

In some embodiments, the present disclosure provides an anti-B7-H3 antibody or antigen-binding fragment thereof, comprising a heavy chain variable region and a light chain variable region, wherein
(1) the heavy chain variable region comprises HCDR1, HCDR2 and HCDR3, selected from the group consisting of:
(a1) amino acid sequences as set forth in SEQ ID NOs: 1, 2 and 3,
(a2) amino acid sequences as set forth in SEQ ID NOs: 7, 8 and 9,
(a3) amino acid sequences as set forth in SEQ ID NOs: 13, 14 and 15,
(a4) amino acid sequences as set forth in SEQ ID NOs: 20, 21 and 22, and
(a5) CDRs having at least 85% sequence identity to the amino acid sequences set forth in (a1), (a2), (a3), or (a4); and
(2) the light chain variable region comprises LCDR1, LCDR2 and LCDR3, selected from the group consisting of:
(a6) amino acid sequences as set forth in SEQ ID NOs: 4, 5 and 6,
(a7) amino acid sequences as set forth in SEQ ID NOs: 10, 11 and 12,
(a8) amino acid sequences as set forth in SEQ ID NOs: 16, 18 and 19,
(a9) amino acid sequences as set forth in SEQ ID NOs: 17, 18 and 19,
(a10) amino acid sequences as set forth in SEQ ID NOs: 23, 24 and 25, and
(a11) CDRs having at least 85% sequence identity to the amino acid sequences set forth in (a6), (a7), (a8), (a9), or (a10).

In a specific embodiment, the present disclosure provides an anti-B7-H3 antibody or antigen-binding fragment thereof, comprising a heavy chain variable region and a light chain variable region, wherein the heavy chain variable region comprises HCDR1, HCDR2, and HCDR3 set forth in SEQ ID NOs: 1, 2 and 3 respectively, or the CDRs having at least 85% sequence identity to the amino acid sequences set forth in SEQ ID NOs: 1, 2 and 3; and the light chain variable region comprises LCDR1, LCDR2 and LCDR3 set forth in SEQ ID NOs: 4, 5 and 6 respectively, or the CDRs having at least 85% sequence identity to the amino acid sequences set forth in SEQ ID NOs: 4, 5 and 6.

In a specific embodiment, the present disclosure provides an anti-B7-H3 antibody or antigen-binding fragment thereof, comprising a heavy chain variable region and a light chain variable region, wherein the heavy chain variable region comprises HCDR1, HCDR2, and HCDR3 set forth in SEQ ID NOs: 7, 8 and 9 respectively, or the CDRs having at least 85% sequence identity to the amino acid sequences set forth in SEQ ID NOs: 7, 8 and 9; and the light chain variable region comprises LCDR1, LCDR2 and LCDR3 set forth in SEQ ID NOs: 10, 11 and 12 respectively, or the CDRs having at least 85% sequence identity to the amino acid sequences set forth in SEQ ID NOs: 10, 11 and 12.

In a specific embodiment, the present disclosure provides an anti-B7-H3 antibody or antigen-binding fragment thereof, comprising a heavy chain variable region and a light chain variable region, wherein the heavy chain variable region comprises HCDR1, HCDR2, and HCDR3 set forth in SEQ ID NOs: 13, 14 and 15 respectively, or the CDRs having at least 85% sequence identity to the amino acid sequences set forth in SEQ ID NOs: 13, 14 and 15; and the light chain variable region comprises LCDR1, LCDR2 and LCDR3 set forth in SEQ ID NOs: 16, 18 and 19 respectively, or the CDRs having at least 85% sequence identity to the amino acid sequences set forth in SEQ ID NOs: 16, 18 and 19.

In a specific embodiment, the present disclosure provides an anti-B7-H3 antibody or antigen-binding fragment thereof, comprising a heavy chain variable region and a light chain variable region, wherein the heavy chain variable region comprises HCDR1, HCDR2, and HCDR3 set forth in SEQ ID NOs: 13, 14 and 15 respectively, or the CDRs having at least 85% sequence identity to the amino acid sequences set forth in SEQ ID NOs: 13, 14 and 15; and the light chain variable region comprises LCDR1, LCDR2 and LCDR3 set forth in SEQ ID NOs: 17, 18 and 19 respectively, or the CDRs having at least 85% sequence identity to the amino acid sequences set forth in SEQ ID NOs: 17, 18 and 19.

In a specific embodiment, the present disclosure provides an anti-B7-H3 antibody or antigen-binding fragment thereof, comprising a heavy chain variable region and a light chain variable region, wherein the heavy chain variable region comprises HCDR1, HCDR2, and HCDR3 set forth in SEQ ID NOs: 20, 21 and 22 respectively, or the CDRs having at least 85% sequence identity to the amino acid sequences set forth in SEQ ID NOs: 20, 21 and 22; and the light chain variable region comprises LCDR1, LCDR2 and LCDR3 set forth in SEQ ID NOs: 23, 24 and 25 respectively, or the CDRs having at least 85% sequence identity to the amino acid sequences set forth in SEQ ID NOs: 23, 24 and 25.

In some specific embodiments, the anti-B7-H3 antibody or antigen-binding fragment thereof according to the present disclosure is a monoclonal antibody or antigen-binding fragment thereof.

In some specific embodiments, the anti-B7-H3 antibody or antigen-binding fragment thereof according to the present disclosure is a murine-derived antibody or antigen-binding fragment thereof, a chimeric antibody or antigen-binding fragment thereof, or a humanized antibody or antigen-binding fragment thereof.

In some specific embodiments, the present disclosure provides an anti-B7-H3 antibody or antigen-binding fragment thereof, comprising a heavy chain variable region and a light chain variable region, wherein
(1) the amino acid sequence of the heavy chain variable region is selected from the group consisting of:
(b1) amino acid sequences as set forth in SEQ ID NO: 26, SEQ ID NO: 28, SEQ ID NO: 30 and SEQ ID NO: 35,
(b2) amino acid sequences derived from the amino acid sequences set forth in (b1) by substitution, deletion or addition of one or more amino acids and functionally identical or similar to the amino acid sequences set forth in (b1), and
(b3) amino acid sequences having at least 80% sequence identity to the amino acid sequences set forth in (b1); and
(2) the amino acid sequence of the light chain variable region is selected from the group consisting of:
(b4) amino acid sequences as set forth in SEQ ID NO: 27, SEQ ID NO: 29, SEQ ID NO: 31, SEQ ID NO: 32 and SEQ ID NO: 36,
(b5) amino acid sequences derived from the amino acid sequences set forth in (b4) by substitution, deletion or addition of one or more amino acids and functionally identical or similar to the amino acid sequences set forth in (b4), and
(b6) amino acid sequences having at least 80% sequence identity to the amino acid sequences set forth in (b4).

In some specific embodiments, the present disclosure provides an anti-B7-H3 antibody or antigen-binding fragment thereof, wherein the amino acid sequence of the heavy chain variable region is selected from the group consisting of SEQ ID NO: 26, the amino acid sequence derived from SEQ ID NO: 26 by substitution, deletion or addition of one or more amino acids and functionally identical to SEQ ID NO: 26, and the amino acid sequence having at least 85% sequence identity to SEQ ID NO: 26; and the amino acid sequence of the light chain variable region is selected from the group consisting of SEQ ID NO: 27, the amino acid sequence derived from SEQ ID NO: 27 by substitution, deletion or addition of one or more amino acids and functionally identical to SEQ ID NO: 27, and the amino acid sequence having at least 85% sequence identity to SEQ ID NO: 27.

In some specific embodiments, the present disclosure provides an anti-B7-H3 antibody or antigen-binding fragment thereof, wherein the amino acid sequence of the heavy chain variable region is selected from the group consisting of SEQ ID NO: 28, the amino acid sequence derived from SEQ ID NO: 28 by substitution, deletion or addition of one or more amino acids and functionally identical to SEQ ID NO: 28, and the amino acid sequence having at least 85% sequence identity to SEQ ID NO: 28; and the amino acid sequence of the light chain variable region is selected from the group consisting of SEQ ID NO: 29, the amino acid sequence derived from SEQ ID NO: 29 by substitution, deletion or addition of one or more amino acids and functionally identical to SEQ ID NO: 29, and the amino acid sequence having at least 85% sequence identity to SEQ ID NO: 29.

In some specific embodiments, the present disclosure provides an anti-B7-H3 antibody or antigen-binding fragment thereof, wherein the amino acid sequence of the heavy chain variable region is selected from the group consisting of SEQ ID NO: 30, the amino acid sequence derived from SEQ ID NO: 30 by substitution, deletion or addition of one or more amino acids and functionally identical to SEQ ID NO: 30, and the amino acid sequence having at least 85% sequence identity to SEQ ID NO: 30; and the amino acid sequence of the light chain variable region is selected from the group consisting of SEQ ID NO: 31, the amino acid sequence derived from SEQ ID NO: 31 by substitution, deletion or addition of one or more amino acids and functionally identical to SEQ ID NO: 31, and the amino acid sequence having at least 85% sequence identity to SEQ ID NO: 31.

In some specific embodiments, the present disclosure provides an anti-B7-H3 antibody or antigen-binding fragment thereof, wherein the amino acid sequence of the heavy chain variable region is selected from the group consisting of SEQ ID NO: 30, the amino acid sequence derived from SEQ ID NO: 30 by substitution, deletion or addition of one or more amino acids and functionally identical to SEQ ID NO: 30, and the amino acid sequence having at least 85% sequence identity to SEQ ID NO: 30; and the amino acid sequence of the light chain variable region is selected from the group consisting of SEQ ID NO: 32, the amino acid sequence derived from SEQ ID NO: 32 by substitution, deletion or addition of one or more amino acids and functionally identical to SEQ ID NO: 32, and the amino acid sequence having at least 85% sequence identity to SEQ ID NO: 32.

In some specific embodiments, the present disclosure provides an anti-B7-H3 antibody or antigen-binding fragment thereof, wherein the amino acid sequence of the heavy chain variable region is selected from the group consisting of SEQ ID NO: 35, the amino acid sequence derived from SEQ ID NO: 35 by substitution, deletion or addition of one or more amino acids and functionally identical to SEQ ID NO: 35, and the amino acid sequence having at least 85% sequence identity to SEQ ID NO: 35; and the amino acid sequence of the light chain variable region is selected from the group consisting of SEQ ID NO: 36, the amino acid sequence derived from SEQ ID NO: 36 by substitution, deletion or addition of one or more amino acids and functionally identical to SEQ ID NO: 36, and the amino acid sequence having at least 85% sequence identity to SEQ ID NO: 36.

In some specific embodiments, the present disclosure provides an anti-B7-H3 antibody or antigen-binding fragment thereof, wherein the amino acid sequence of the heavy chain variable region is selected from the group consisting of SEQ ID NO: 26, the amino acid sequence derived from SEQ ID NO: 26 by substitution, deletion or addition of one or more amino acids and functionally identical to SEQ ID NO: 26, and the amino acid sequence having at least 85% sequence identity to SEQ ID NO: 26, and comprises HCDR1, HCDR2, and HCDR3 set forth in SEQ ID NOs: 1, 2 and 3; and the amino acid sequence of the light chain variable region is selected from the group consisting of SEQ ID NO: 27, the amino acid sequence derived from SEQ ID NO: 27 by substitution, deletion or addition of one or more amino acids and functionally identical to SEQ ID NO: 27, and the amino acid sequence having at least 85% sequence identity to SEQ ID NO: 27, and comprises LCDR1, LCDR2 and LCDR3 set forth in SEQ ID NOs: 4, 5 and 6.

In some specific embodiments, the present disclosure provides an anti-B7-H3 antibody or antigen-binding fragment thereof, wherein the amino acid sequence of the heavy chain variable region is selected from the group consisting of SEQ ID NO: 28, the amino acid sequence derived from SEQ ID NO: 28 by substitution, deletion or addition of one or more amino acids and functionally identical to SEQ ID NO: 28, and the amino acid sequence having at least 85% sequence identity to SEQ ID NO: 28, and comprises HCDR1, HCDR2, and HCDR3 set forth in SEQ ID NOs: 7, 8 and 9; and the amino acid sequence of the light chain variable region is selected from the group consisting of SEQ ID NO: 29, the amino acid sequence derived from SEQ ID NO: 29 by substitution, deletion or addition of one or more amino acids and functionally identical to SEQ ID NO: 29, and the amino acid sequence having at least 85% sequence identity to SEQ ID NO: 29, and comprises LCDR1, LCDR2 and LCDR3 set forth in SEQ ID NOs: 10, 11 and 12.

In some specific embodiments, the present disclosure provides an anti-B7-H3 antibody or antigen-binding fragment thereof, wherein the amino acid sequence of the heavy chain variable region is selected from the group consisting of SEQ ID NO: 30, the amino acid sequence derived from SEQ ID NO: 30 by substitution, deletion or addition of one or more amino acids and functionally identical to SEQ ID NO: 30, and the amino acid sequence having at least 85% sequence identity to SEQ ID NO: 30, and comprises HCDR1, HCDR2, and HCDR3 set forth in SEQ ID NOs: 13, 14 and 15; and the amino acid sequence of the light chain variable region is selected from the group consisting of SEQ ID NO: 31, the amino acid sequence derived from SEQ ID NO: 31 by substitution, deletion or addition of one or more amino acids and functionally identical to SEQ ID NO: 31, and the amino acid sequence having at least 85% sequence identity to SEQ ID NO: 31, and comprises LCDR1, LCDR2 and LCDR3 set forth in SEQ ID NOs: 16, 18 and 19.

In some specific embodiments, the present disclosure provides an anti-B7-H3 antibody or antigen-binding fragment thereof, wherein the amino acid sequence of the heavy chain variable region is selected from the group consisting of SEQ ID NO: 30, the amino acid sequence derived from SEQ ID NO: 30 by substitution, deletion or addition of one or more amino acids and functionally identical to SEQ ID NO: 30, and the amino acid sequence having at least 85% sequence identity to SEQ ID NO: 30, and comprises HCDR1, HCDR2, and HCDR3 set forth in SEQ ID NOs: 13, 14 and 15; and the amino acid sequence of the light chain variable region is selected from the group consisting of SEQ ID NO: 32, the amino acid sequence derived from SEQ ID NO: 32 by substitution, deletion or addition of one or more amino acids and functionally identical to SEQ ID NO: 32, and the amino acid sequence having at least 85% sequence identity to SEQ ID NO: 32, and comprises LCDR1, LCDR2 and LCDR3 set forth in SEQ ID NOs: 17, 18 and 19.

In some specific embodiments, the present disclosure provides an anti-B7-H3 antibody or antigen-binding fragment thereof, wherein the amino acid sequence of the heavy chain variable region is selected from the group consisting of SEQ ID NO: 35, the amino acid sequence derived from SEQ ID NO: 35 by substitution, deletion or addition of one or more amino acids and functionally identical to SEQ ID NO: 35, and the amino acid sequence having at least 85% sequence identity to SEQ ID NO: 35, and comprises HCDR1, HCDR2, and HCDR3 set forth in SEQ ID NOs: 20, 21 and 22; and the amino acid sequence of the light chain variable region is selected from the group consisting of SEQ ID NO: 36, the amino acid sequence derived from SEQ ID NO: 36 by substitution, deletion or addition of one or more amino acids and functionally identical to SEQ ID NO: 36, and the amino acid sequence having at least 85% sequence identity to SEQ ID NO: 36, and comprises LCDR1, LCDR2 and LCDR3 set forth in SEQ ID NOs: 23, 24 and 25.

In some specific embodiments, the anti-B7-H3 antibody according to the present disclosure is a murine-derived antibody, which further comprises a heavy chain constant region of murine-derived IgG1, IgG2, IgG3, IgG4, or a variant thereof, and a light chain constant region of murine-derived kappa chain or a variant thereof.

In some preferred embodiments, the anti-B7-H3 murine-derived antibody according to the present disclosure further comprises a heavy chain constant region of murine-derived IgG1, IgG2, or a variant thereof, and a light chain constant region of murine-derived kappa chain or a variant thereof.

In some embodiments, the present disclosure provides an anti-B7-H3 antibody or antigen-binding fragment thereof, wherein the anti-B7-H3 antibody is a humanized antibody, wherein
(1) the amino acid sequence of the heavy chain variable region is selected from the group consisting of:
(c1) amino acid sequences as set forth in SEQ ID NO: 45, SEQ ID NO: 46, SEQ ID NO: 47, SEQ ID NO: 48, SEQ ID NO: 52, SEQ ID NO: 53, SEQ ID NO: 54, SEQ ID NO: 55, SEQ ID NO: 56, SEQ ID NO: 57, SEQ ID NO: 63, SEQ ID NO: 64, SEQ ID NO: 72 and SEQ ID NO: 73,
(c2) amino acid sequences derived from the amino acid sequences set forth in (c1) by substitution, deletion or addition of one or more amino acids and functionally identical or similar to the amino acid sequences as set forth in (c1), and
(c3) amino acid sequences having at least 80% sequence identity to the amino acid sequences set forth in (c1); and
(2) the amino acid sequence of the light chain variable region is selected from the group consisting of:
(c4) amino acid sequences as set forth in SEQ ID NO: 49, SEQ ID NO: 50, SEQ ID NO: 51, SEQ ID NO: 58, SEQ ID NO: 59, SEQ ID NO: 60, SEQ ID NO: 61, SEQ ID NO: 62, SEQ ID NO: 65, SEQ ID NO: 66, SEQ ID NO: 67, SEQ ID NO: 68, SEQ ID NO: 69, SEQ ID NO: 70, SEQ ID NO: 74, SEQ ID NO: 75, SEQ ID NO: 76, SEQ ID NO: 77, SEQ ID NO: 78 and SEQ ID NO: 79,
(c5) amino acid sequences derived from the amino acid sequences set forth in (c4) by substitution, deletion or addition of one or more amino acids and functionally identical or similar to the amino acid sequences as set forth in (c4), and
(c6) amino acid sequences having at least 80% sequence identity to the amino acid sequences set forth in (c4).

In some specific embodiments, the present disclosure provides an anti-B7-H3 antibody or antigen-binding fragment thereof, wherein the anti-B7-H3 antibody is a humanized antibody, wherein
(1) the amino acid sequence of the heavy chain variable region is selected from the group consisting of:
(c1) amino acid sequences as set forth in SEQ ID NO: 45, SEQ ID NO: 46, SEQ ID NO: 47 and SEQ ID NO: 48,
(c2) amino acid sequences derived from the amino acid sequences set forth in (c1) by substitution, deletion or addition of one or more amino acids and functionally identical or similar to the amino acid sequences as set forth in (c1), and
(c3) amino acid sequences having at least 85% sequence identity to the amino acid sequences set forth in (c1) and comprising HCDR1, HCDR2 and HCDR3 set forth in SEQ ID NOs: 1, 2 and 3; and
(2) the amino acid sequence of the light chain variable region is selected from the group consisting of:
(c4) amino acid sequences as set forth in SEQ ID NO: 49, SEQ ID NO: 50 and SEQ ID NO: 51,
(c5) amino acid sequences derived from the amino acid sequences set forth in (c4) by substitution, deletion or addition of one or more amino acids and functionally identical or similar to the amino acid sequences as set forth in (c4), and
(c6) amino acid sequences having at least 85% sequence identity to the amino acid sequences set forth in (c4) and comprising LCDR1, LCDR2 and LCDR3 set forth in SEQ ID NOs: 4, 5 and 6.

In some specific embodiments, the present disclosure provides an anti-B7-H3 antibody or antigen-binding fragment thereof, wherein the anti-B7-H3 antibody is a humanized antibody, wherein
(1) the amino acid sequence of the heavy chain variable region is selected from the group consisting of:
(c1) amino acid sequences as set forth in SEQ ID NO: 52, SEQ ID NO: 53, SEQ ID NO: 54, SEQ ID NO: 55, SEQ ID NO: 56 and SEQ ID NO: 57,
(c2) amino acid sequences derived from the amino acid sequences set forth in (c1) by substitution, deletion or addition of one or more amino acids and functionally identical or similar to the amino acid sequences as set forth in (c1), and
(c3) amino acid sequences having at least 85% sequence identity to the amino acid sequences set forth in (c1) and comprising HCDR1, HCDR2 and HCDR3 set forth in SEQ ID NOs: 7, 8 and 9; and
(2) the amino acid sequence of the light chain variable region is selected from the group consisting of:
(c4) amino acid sequences as set forth in SEQ ID NO: 58, SEQ ID NO: 59, SEQ ID NO: 60, SEQ ID NO: 61 and SEQ ID NO: 62,
(c5) amino acid sequences derived from the amino acid sequences set forth in (c4) by substitution, deletion or addition of one or more amino acids and functionally identical or similar to the amino acid sequences as set forth in (c4), and
(c6) amino acid sequences having at least 85% sequence identity to the amino acid sequences set forth in (c4) and comprising LCDR1, LCDR2 and LCDR3 set forth in SEQ ID NOs: 10, 11 and 12.

In some specific embodiments, the present disclosure provides an anti-B7-H3 antibody or antigen-binding fragment thereof, wherein the anti-B7-H3 antibody is a humanized antibody, wherein
(1) the amino acid sequence of the heavy chain variable region is selected from the group consisting of:
(c1) amino acid sequences as set forth in SEQ ID NO: 63 and SEQ ID NO: 64,
(c2) amino acid sequences derived from the amino acid sequences set forth in (c1) by substitution, deletion or addition of one or more amino acids and functionally identical or similar to the amino acid sequences as set forth in (c1), and
(c3) amino acid sequences having at least 85% sequence identity to the amino acid sequences set forth in (c1) and comprising HCDR1, HCDR2 and HCDR3 set forth in SEQ ID NOs: 13, 14 and 15; and
(2) the amino acid sequence of the light chain variable region is selected from the group consisting of:
(c4) amino acid sequences as set forth in SEQ ID NO: 65, SEQ ID NO: 66, SEQ ID NO: 67, SEQ ID NO: 68, SEQ ID NO: 69 and SEQ ID NO: 70,
(c5) amino acid sequences derived from the amino acid sequences set forth in (c4) by substitution, deletion or addition of one or more amino acids and functionally identical or similar to the amino acid sequences as set forth in (c4), and
(c6) amino acid sequences having at least 85% sequence identity to the amino acid sequences set forth in (c4) and comprising LCDR1, LCDR2 and LCDR3 set forth in SEQ ID NOs: 17, 18 and 19.

In some specific embodiments, the present disclosure provides an anti-B7-H3 antibody or antigen-binding fragment thereof, wherein the anti-B7-H3 antibody is a humanized antibody, wherein
(1) the amino acid sequence of the heavy chain variable region is selected from the group consisting of:
(c1) amino acid sequences as set forth in SEQ ID NO: 72 and SEQ ID NO: 73,
(c2) amino acid sequences derived from the amino acid sequences set forth in (c1) by substitution, deletion or addition of one or more amino acids and functionally identical or similar to the amino acid sequences as set forth in (c1), and
(c3) amino acid sequences having at least 85% sequence identity to the amino acid sequences set forth in (c1) and comprising HCDR1, HCDR2 and HCDR3 set forth in SEQ ID NOs: 20, 21 and 22; and
(2) the amino acid sequence of the light chain variable region is selected from the group consisting of:
(c4) amino acid sequences as set forth in SEQ ID NO: 74, SEQ ID NO: 75, SEQ ID NO: 76, SEQ ID NO: 77, SEQ ID NO: 78 and SEQ ID NO: 79,
(c5) amino acid sequences derived from the amino acid sequences set forth in (c4) by substitution, deletion or addition of one or more amino acids and functionally identical or similar to the amino acid sequences as set forth in (c4), and
(c6) amino acid sequences having at least 85% sequence identity to the amino acid sequences set forth in (c4) and comprising LCDR1, LCDR2 and LCDR3 set forth in SEQ ID NOs: 23, 24 and 25.

In some specific embodiments, the present disclosure provides an anti-B7-H3 antibody or antigen-binding fragment thereof, wherein the amino acid sequence of the heavy chain variable region is selected from the group consisting of SEQ ID NO: 45, the amino acid sequence derived from SEQ ID NO: 45 by substitution, deletion or addition of one or more amino acids and functionally identical to SEQ ID NO: 45, and the amino acid sequence having at least 85% sequence identity to SEQ ID NO: 45 and comprising HCDR1, HCDR2 and HCDR3 set forth in SEQ ID NOs: 1, 2 and 3; and the amino acid sequence of the light chain variable region is selected from the group consisting of SEQ ID NO: 49, the amino acid sequence derived from SEQ ID NO: 49 by substitution, deletion or addition of one or more amino acids and functionally identical to SEQ ID NO: 49, and the amino acid sequence having at least 85% sequence identity to SEQ ID NO: 49 and comprising LCDR1, LCDR2 and LCDR3 set forth in SEQ ID NOs: 4, 5 and 6.

In some specific embodiments, the present disclosure provides an anti-B7-H3 antibody or antigen-binding fragment thereof, wherein the amino acid sequence of the heavy chain variable region is selected from the group consisting of SEQ ID NO: 52, the amino acid sequence derived from SEQ ID NO: 52 by substitution, deletion or addition of one or more amino acids and functionally identical to SEQ ID NO: 52, and the amino acid sequence having at least 85% sequence identity to SEQ ID NO: 52 and comprising HCDR1, HCDR2 and HCDR3 set forth in SEQ ID NOs: 7, 8 and 9; and the amino acid sequence of the light chain variable region is selected from the group consisting of SEQ ID NO: 59, the amino acid sequence derived from SEQ ID NO: 59 by substitution, deletion or addition of one or more amino acids and functionally identical to SEQ ID NO: 59, and the amino acid sequence having at least 85% sequence identity to SEQ ID NO: 59 and comprising LCDR1, LCDR2 and LCDR3 set forth in SEQ ID NOs: 10, 11 and 12.

In some specific embodiments, the present disclosure provides an anti-B7-H3 antibody or antigen-binding fragment thereof, wherein the amino acid sequence of the heavy chain variable region is selected from the group consisting of SEQ ID NO: 63, the amino acid sequence derived from SEQ ID NO: 63 by substitution, deletion or addition of one or more amino acids and functionally identical to SEQ ID NO: 63, and the amino acid sequence having at least 85% sequence identity to SEQ ID NO: 63 and comprising HCDR1, HCDR2 and HCDR3 set forth in SEQ ID NOs: 13, 14 and 15; and the amino acid sequence of the light chain variable region is selected from the group consisting of SEQ ID NO: 65, the amino acid sequence derived from SEQ ID NO: 65 by substitution, deletion or addition of one or more amino acids and functionally identical to SEQ ID NO: 65, and the amino acid sequence having at least 85% sequence identity to SEQ ID NO: 65 and comprising LCDR1, LCDR2 and LCDR3 set forth in SEQ ID NOs: 17, 18 and 19.

In some specific embodiments, the present disclosure provides an anti-B7-H3 antibody or antigen-binding fragment thereof, wherein the amino acid sequence of the heavy chain variable region is selected from the group consisting of SEQ ID NO: 64, the amino acid sequence derived from SEQ ID NO: 64 by substitution, deletion or addition of one or more amino acids and functionally identical to SEQ ID NO: 64, and the amino acid sequence having at least 85% sequence identity to SEQ ID NO: 64 and comprising HCDR1, HCDR2 and HCDR3 set forth in SEQ ID NOs: 13, 14 and 15; and the amino acid sequence of the light chain variable region is selected from the group consisting of SEQ ID NO: 67, the amino acid sequence derived from SEQ ID NO: 67 by substitution, deletion or addition of one or more amino acids and functionally identical to SEQ ID NO: 67, and the amino acid sequence having at least 85% sequence identity to SEQ ID NO: 67 and comprising LCDR1, LCDR2 and LCDR3 set forth in SEQ ID NOs: 17, 18 and 19.

In some specific embodiments, the present disclosure provides an anti-B7-H3 antibody or antigen-binding fragment thereof, wherein the amino acid sequence of the heavy chain variable region is selected from the group consisting of SEQ ID NO: 72, the amino acid sequence derived from SEQ ID NO: 72 by substitution, deletion or addition of one or more amino acids and functionally identical to SEQ ID NO: 72, and the amino acid sequence having at least 85% sequence identity to SEQ ID NO: 72 and comprising HCDR1, HCDR2 and HCDR3 set forth in SEQ ID NOs: 20, 21 and 22; and the amino acid sequence of the light chain variable region is selected from the group consisting of SEQ ID NO: 77, the amino acid sequence derived from SEQ ID NO: 77 by substitution, deletion or addition of one or more amino acids and functionally identical to SEQ ID NO: 77, and the amino acid sequence having at least 85% sequence identity to SEQ ID NO: 77 and comprising LCDR1, LCDR2 and LCDR3 set forth in SEQ ID NOs: 23, 24 and 25.

In some specific embodiments, the present disclosure provides an anti-B7-H3 antibody or antigen-binding fragment thereof, wherein the amino acid sequence of the heavy chain variable region is selected from the group consisting of SEQ ID NO: 22, the amino acid sequence derived from SEQ ID NO: 22 by substitution, deletion or addition of one or more amino acids and functionally identical to SEQ ID NO: 22, and the amino acid sequence having at least 85% sequence identity to SEQ ID NO: 22 and comprising HCDR1, HCDR2 and HCDR3 set forth in SEQ ID NOs: 1, 2 and 3; and the amino acid sequence of the light chain variable region is selected from the group consisting of SEQ ID NO: 25, the amino acid sequence derived from SEQ ID NO: 25 by substitution, deletion or addition of one or more amino acids and functionally identical to SEQ ID NO: 25, and the amino acid sequence having at least 85% sequence identity to SEQ ID NO: 25 and comprising LCDR1, LCDR2 and LCDR3 set forth in SEQ ID NOs: 10, 11 and 12.

In some embodiments, the present disclosure provides an anti-B7-H3 humanized antibody or antigen-binding fragment thereof, wherein the heavy chain comprises a heavy chain constant region of humanized IgG1, IgG2, IgG3, IgG4 or a variant thereof, and the light chain comprises a light chain constant region of humanized kappa, lambda chain, or a variant thereof.

In a preferred embodiment of the present disclosure, the murine-derived anti-B7-H3 antibody or antigen-binding fragment thereof may further comprise a light chain constant region of murine-derived kappa, lambda chain, or a variant thereof, and/or further comprise a heavy chain constant region of murine-derived IgG1, IgG2, IgG3, IgG4, or a variant thereof.

In a preferred embodiment of the present disclosure, the antibody light chain of the anti-B7-H3 chimeric antibody or antigen-binding fragment thereof further comprises a light chain constant region of murine-derived kappa, lambda chain, or mutant sequences thereof. The antibody heavy chain of the anti-B7-H3 chimeric antibody or antigen-binding fragment thereof further comprises a heavy chain constant region of murine-derived IgG1, IgG2, IgG3, IgG4, or mutant sequences thereof, and preferably comprises a heavy chain constant region of humanized IgG1 or IgG2, or IgG4 constant region that significantly reduces ADCC (antibody-dependent cell-mediated cytotoxicity) after amino acid mutation.

In some specific embodiments, the anti-B7-H3 humanized antibody or antigen-binding fragment thereof of the present disclosure further comprises a heavy chain constant region of humanized IgG1, IgG2, IgG3, IgG4, or a variant thereof, and a light chain constant region of humanized kappa chain, lambda chain, or a variant thereof. In some preferred embodiments, the anti-B7-H3 humanized antibody or antigen-binding fragment thereof of the present disclosure further comprises a heavy chain constant region of humanized IgG1, IgG2, or a variant thereof, and a light chain constant region of humanized kappa chain or a variant thereof.

In some embodiments, the present disclosure provides an anti-B7-H3 antibody or antigen-binding fragment thereof, wherein the antigen-binding fragment is Fab, Fv, sFv or F(ab)₂.

Another aspect of the present disclosure provides an isolated nucleic acid encoding the anti-B7-H3 antibody or antigen-binding fragment thereof according to the present disclosure.

In some specific embodiments, the isolated nucleic acid according to the present disclosure, wherein the nucleotide sequence encoding the amino acid sequence of the heavy chain variable region is selected from the group consisting of SEQ ID NO: 80, SEQ ID NO: 82, SEQ ID NO: 84 and SEQ ID NO: 86; and the nucleotide sequence encoding the amino acid sequence of the light chain variable region is selected from the group consisting of SEQ ID NO: 81, SEQ ID NO: 83, SEQ ID NO: 85 and SEQ ID NO: 87.

In a specific embodiment, the isolated nucleic acid according to the present disclosure, wherein the nucleotide sequence encoding the heavy chain variable region SEQ ID NO: 45 is set forth in SEQ ID NO: 80; the nucleotide sequence encoding the light chain variable region SEQ ID NO: 49 is set forth in SEQ ID NO: 81.

In a specific embodiment, the isolated nucleic acid according to the present disclosure, wherein the nucleotide sequence encoding the heavy chain variable region SEQ ID NO: 52 is set forth in SEQ ID NO: 82; the nucleotide sequence encoding the light chain variable region SEQ ID NO: 59 is set forth in SEQ ID NO: 83.

In a specific embodiment, the isolated nucleic acid according to the present disclosure, wherein the nucleotide sequence encoding the heavy chain variable region SEQ ID NO: 64 is set forth in SEQ ID NO: 84; the nucleotide sequence encoding the light chain variable region SEQ ID NO: 67 is set forth in SEQ ID NO: 85.

In a specific embodiment, the isolated nucleic acid according to the present disclosure, wherein the nucleotide sequence encoding the heavy chain variable region SEQ ID NO: 72 is set forth in SEQ ID NO: 86; the nucleotide sequence encoding the light chain variable region SEQ ID NO: 77 is set forth in SEQ ID NO: 87.

Another aspect of the present disclosure provides an expression vector expressing the anti-B7-H3 antibody or antigen-binding fragment thereof of the present disclosure. The expression vector according to the present disclosure comprises the isolated nucleic acid molecule of the present disclosure.

Another aspect of the present disclosure provides a host cell transformed with the expression vector as described above.

In some embodiments, the host cell according to the present disclosure is selected from the group consisting of prokaryotic cells and eukaryotic cells. In some embodiments, the host cell is bacteria, preferably Escherichia coli. In another preferred embodiment, the host cell is mammalian cells.

Another aspect of the present disclosure provides a method for producing the anti-B7-H3 antibody or antigen-binding fragment thereof of the present disclosure, comprising expressing the antibody in the host cell and isolating the antibody from the host cell.

Another aspect of the present disclosure provides a pharmaceutical composition comprising the anti-B7-H3 humanized antibody or antigen-binding fragment thereof of the present disclosure and a pharmaceutically acceptable carrier. In some embodiments, the present disclosure provides a pharmaceutical composition comprising the anti-B7-H3 humanized antibody or antigen-binding fragment thereof of the present disclosure, and also other active components, such as other antibodies, targeting drugs, and the like. In some embodiments, the pharmaceutically acceptable carrier is selected from the group consisting of antioxidants, polypeptides, proteins, hydrophilic polymers, amino acids, saccharides, chelating agents, alditols, ions, and surfactants. In a specific embodiment, the pharmaceutically acceptable carrier is a buffered aqueous solution. In another specific embodiment, the pharmaceutically acceptable carrier is in the form of liposomes.

The anti-B7-H3 humanized antibody or antigen-binding fragment thereof of the present disclosure can be combined with a pharmaceutically acceptable carrier, diluent or excipient to prepare a pharmaceutical preparation suitable for oral or parenteral administration. The routes of administration include, but are not limited to oral, intradermal, intramuscular, intraperitoneal, intravenous, intracerebral, intraocular, intratracheal, subcutaneous, and intranasal routes. The preparation can be administered by any means, for example, by infusion or bolus injection, by the means of absorption through epithelium or skin mucosa (for example, oral mucosa or rectum, etc.). Administration can be systemic or local. The preparation can be prepared by methods known in the art, and contains a carrier, diluent or excipient conventionally used in the field of pharmaceutical preparations.

Another aspect of the present disclosure provides a method for inhibiting B7-H3 activity, comprising administering the anti-B7-H3 antibody or antigen-binding fragment thereof of the present disclosure or the pharmaceutical composition of the present disclosure to a subject in need thereof.

Another aspect of the present disclosure provides use of the anti-B7-H3 antibody or antigen-binding fragment thereof of the present disclosure or the pharmaceutical composition of the present disclosure in the manufacture of a medicament for inhibiting B7-H3 activity. In some embodiments, the medicament for inhibiting B7-H3 activity is used to treat leukemia, lymphoma, breast cancer, lung cancer, gastric cancer, intestinal cancer, esophageal cancer, ovarian cancer, cervical cancer, kidney cancer, bladder cancer, pancreatic cancer, glioma and/or melanoma. In some embodiments, the present disclosure provides use of the above-mentioned anti-B7-H3 antibody or antigen-binding fragment thereof or the pharmaceutical composition of the present disclosure in the manufacture of anti-tumor drugs. Preferably, the tumor is selected from the group consisting of leukemia, lymphoma, breast cancer, lung cancer, gastric cancer, intestinal cancer, esophageal cancer, ovarian cancer, kidney cancer, bladder cancer, pancreatic cancer, glioma and melanoma.

The anti-B7-H3 antibody or antigen-binding fragment thereof provided by the present disclosure has a higher affinity and stability and significant anti-tumor effect, and can significantly inhibit tumor growth. The humanized antibody, whose immunogenicity is greatly reduced, effectively eliminates the rejection of exogenous monoclonal antibodies by human immune system and can be used in the manufacture of medicaments for the treatment of various tumor diseases with broad market prospects.

### Definitions

Unless otherwise defined, the meanings of scientific and technical terms used herein are those commonly understood by those skilled in the art. The nomenclature and techniques used in cell and tissue culture, molecular biology, as well as protein and oligo/polynucleotide chemistry and hybridization described herein are well known and commonly used in the art. Standard techniques are used for recombinant DNA, oligonucleotide synthesis, tissue culture and transformation (e.g. electroporation, lipofection). The enzymatic reaction and purification techniques are carried out according to the manufacturer's instructions or methods commonly used in the art or described herein. The aforementioned techniques and methods are generally used according to what's well known in the art and what's described in the multiple comprehensive and more specific documents cited and discussed in this specification. Reference could be made to, Sambrook et al., Molecular Cloning: A Laboratory Manual (2nd edition, Cold Spring Harbor Laboratory Press, Cold Spring Harbor, N.Y. (1989)). The nomenclature as well as laboratory methods and techniques used in analytical chemistry, synthetic organic chemistry, and medical and pharmaceutical chemistry described herein are well known and commonly used in the art.

In the present disclosure, the term “at least 80% sequence identity” refers to at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity. In the present disclosure, the term “at least 85% sequence identity” refers to at least 85%, 86%, 87%, 88%, 89%, 90%, 91%, 92%, 93%, 94%, 95%, 96%, 97%, 98%, 99%, or 100% sequence identity. In some preferred embodiments, the sequence identity described in the present disclosure may be at least 90%, 91%, 92%, 93%, 94%, 95%, 96%, 97%, 98%, 99%, or 100%. Sequence comparison and determination of percent identity between two sequences can be performed by the BLASTN/BLASTP algorithm on the website of National Center For Biotechnology Institute.

In an antibody molecule, three hypervariable regions of the light chain and three hypervariable regions of the heavy chain are arranged relative to each other in a three-dimensional space to form an antigen-binding surface. The antigen-binding surface is complementary to the three-dimensional surface of the bound antigen, and the three hypervariable regions of each heavy chain and light chain are referred as “complementarity determining region” or “CDR”. The assignment of amino acids to each domain is defined by Kabat “Sequences of Proteins of Immunological Interest” (National Institutes of Health, Bethesda, Md. (1987 and 1991)) or Chothia and Lesk (J. Mol. Biol. 196:901-917 (1987), Chothia et al., Nature 342:878-883 (1989)).

The “antigen-binding fragment” of the present disclosure refers to following fragment with antigen-binding activity: Fab fragment, Fab′ fragment, F(ab′)2 fragment, and Fv fragment and scFv fragment that bind to human B7-H3. The Fv fragment comprises a heavy chain variable region and a light chain variable region of the antibody but no constant region, and it is the smallest antibody fragment with all antigen binding sites. Generally, Fv antibody also comprises a polypeptide linker between the VH and VL domains, and is able to form the structure required for antigen binding. Different linkers may also be utilized to link the two variable regions of antibody to form a polypeptide chain, which is called single-chain antibody or single-chain Fv (scFv).

The antibody of the present disclosure refers to an immunoglobulin molecule or an immunologically active part thereof, that is, a molecule that contains antigen-binding sites that specifically bind to the antigen (through immunological reaction). “Specific binding” means that an antibody reacts with one or more antigenic determinants of an antigen but does not react with other polypeptides, or it binds to other polypeptides with very low affinity (Kd>10⁻⁶). Antibodies include but are not limited to polyclonal, monoclonal, chimeric, dAb (domain antibody), single chain, Fab, Fab′ and F(ab′)₂ fragment, Fv, scFv and Fab expression library. A monoclonal antibody (mAb) is the antibody obtained from a single cloned cell line, and the said cell line is not limited to eukaryotic, prokaryotic or phage cloned cell lines. A monoclonal antibody or antigen-binding fragment thereof can be obtained by recombination using, for example, hybridoma technology, recombination technology, phage display technology, and synthesis technology such as CDR grafting, or other existing technology.

The “murine-derived antibody” of the present disclosure is a monoclonal antibody against human B7-H3 produced according to the knowledge and skills in the art. During the production, the test subject is injected with the B7-H3 antigen, and then the hybridomas expressing the antibody with the desired sequence or functional property are isolated.

The “chimeric antibody” of the present disclosure is an antibody formed by fusing the variable regions of a murine-derived antibody with the constant regions of a human antibody, which can reduce the immune response induced by the murine-derived antibody. To establish a chimeric antibody, it is necessary to establish a hybridoma secreting murine-derived specific monoclonal antibodies first, clone the variable region genes from the mouse hybridoma cells, and then clone the constant region genes of the human antibody as needed, and chimeric genes formed by linking the mouse variable region with the human constant region genes are inserted into a human vector. Finally, the chimeric antibody is expressed in a eukaryotic system or a prokaryotic system.

The “humanized antibody” of the present disclosure is also called a CDR grafted antibody, which is the antibody produced by grafting mouse CDR sequences into the variable region framework (FR) of a human antibody. Such variable region framework sequences can be obtained from public DNA databases or public references, for example, from the ImMunoGeneTics (IMGT) website http://imgt.cines.fr or from the Journal of Immunoglobulin, 2001ISBN012441351.

### BRIEF DESCRIPTION OF DRAWINGS

FIG. 1 shows the results of the binding activity assay (ELISA) of the anti-B7-H3 humanized antibody to human 4Ig-B7-H3, wherein the abscissa represents the antibody concentration ng/ml and the ordinate represents the OD450 value.

FIG. 2 shows the results of the binding activity assay (ELISA) of the anti-B7-H3 humanized antibody to human 2Ig-B7-H3, wherein the abscissa represents the antibody concentration ng/ml and the ordinate represents the OD450 value.

FIG. 3 shows the results of the binding activity assay (ELISA) of the anti-B7-H3 humanized antibody to monkey B7-H3, wherein the abscissa represents the antibody concentration ng/ml and the ordinate represents the OD450 value.

FIG. 4 shows the detection results of IFNγ release level after anti-human B7-H3 murine monoclonal antibody treatment in SEB-stimulated PBMC system, wherein the PBMC is from donor B. The abscissa represents the different antibodies, and the ordinate represents the amount of IFNγ released (pg/ml).

FIG. 5 shows the detection results of IFNγ release level after anti-human B7-H3 murine monoclonal antibody treatment in SEB-stimulated PBMC system, wherein the PBMC is from donor C. The abscissa represents the different antibodies, and the ordinate represents the amount of IFNγ released (pg/ml).

FIG. 6 shows the detection results of IFNγ release level after anti-B7-H3 humanized antibody hu65 treatment in SEB-stimulated PBMC system. The abscissa represents the different antibodies, and the ordinate represents the amount of IFNγ released (pg/ml).

FIG. 7 shows the detection results of IFNγ release level after anti-B7-H3 humanized antibody hu51-G34K treatment in SEB-stimulated PBMC system. The abscissa represents the different antibodies, and the ordinate represents the amount of IFNγ released (pg/ml).

### DETAILED DESCRIPTION

The following representative examples are used to illustrate the present disclosure better, rather than to limit the scope of protection of the present disclosure. The experimental methods without conditions indicated in the following examples are usually carried out according to conventional conditions, such as the antibody technology experiment manual and molecular cloning manual of Cold Spring Harbor, or according to the conditions recommended by the raw material or commodity manufacturers. The materials and reagents used in the examples are all commercially available unless otherwise specified.

### Example 1 Preparation of Antigen Protein and Positive Control Antibody

### 1. Construction of Expression Vector of Antigen Protein and Positive Control Antibody

### (1) Construction of Expression Vector of Antigen Protein

A gene fragment encoding the full-length extracellular region of the 4Ig-B7-H3 protein was synthesized, and the amino acid sequence is shown in SEQ ID NO: 88. A gene fragment encoding the 2Ig-B7-H3 protein was synthesized, and the amino acid sequence is shown in SEQ ID NO: 89. The full-length DNA sequence of 4Ig-B7-H3 (SEQ ID NO: 90) was cloned into the eukaryotic expression plasmid pTargeT to generate the expression plasmid pTargeT-4Ig-B7-H3.

The amino acid sequence of the extracellular region of human 4Ig-B7-H3 protein was fused with the amino acid sequence of hIgG1-Fc or his-tag, and the amino acid sequences are shown in SEQ ID NO: 91 and SEQ ID NO: 92. The amino acid sequence of the extracellular region of human 2Ig-B7-H3 protein was fused with the amino acid sequence of hIgG1-Fc or his-tag, and the amino acid sequences are shown in SEQ ID NO: 93 and SEQ ID NO: 94. After codon optimization of the above amino acid sequences, the tagged B7-H3 protein extracellular region coding fragments, 4Ig-B7-H3-hFc, 4Ig-B7-H3-his, 2Ig-B7-H3-hFc, 2Ig-B7-H3-his, were synthesized and cloned into the eukaryotic expression plasmid pHR respectively to generate the respective expression plasmids of the above-mentioned tagged proteins, pHR-4Ig-B7-H3-hFc, pHR-4Ig-B7-H3-his, pHR-2Ig-B7-H3-hFc and pHR-2Ig-B7-H3-his.

(2) Construction of Expression Vector of Positive Control Antibody hBRCA84D

The humanized antibody hBRCA84D disclosed in the patent application WO2011/109400 was used as a positive control antibody, and the amino acid sequences of hBRCA84D are as follows:
heavy chain amino acid sequence of hBRCA84D: SEQ ID NO: 95; and
light chain amino acid sequence of hBRCA84D: SEQ ID NO:96.

hBRCA84D was prepared according to the method disclosed in WO2011/109400. hBRCA84D is a human IgG1 monoclonal antibody. Compared with natural IgG1, its Fc fragment contains specific mutation sites (K214R, L235V, F243L, R292P, Y300L, D356E, L358M and P396L). The amino acid sequences of hBRCA84D humanized antibody were artificially codon-optimized to obtain the full-length DNA sequences of the heavy chain and light chain of the positive control antibody hBRCA84D. The full-length DNA fragment of the hBRCA84D heavy chain was cloned into pHR vector to obtain eukaryotic expression plasmid pHR-hBRCA84D-8mIgG1. The full-length DNA fragment of the hBRCA84D light chain was cloned into pHR vector to obtain eukaryotic expression plasmid pHR-hBRCA84D-hK.

### (3) Construction of Expression Vector for Murine-Derived Positive Control Antibody BRCA84D

The heavy chain variable region of the positive control antibody hBRCA84D was cloned into the eukaryotic expression plasmid pHR-mG2a containing murine mG2a heavy chain constant region to obtain the eukaryotic expression plasmid of pHR-hBRCA84D-mG2a. The light chain variable region of the positive control antibody hBRCA84D was cloned into the eukaryotic expression plasmid pHR-mK containing murine K light chain constant region to obtain the eukaryotic expression plasmid of pHR-hBRCA84D-mK.

### 2. Expression and Purification of Antigen Protein and Positive Control Antibody

### (1) Construction of Stable Transgenic Cell Line Expressing Antigen Protein

CHO-K1 cells (Shanghai Institute of Biochemistry and Cell Biology, Chinese Academy of Sciences) were electrotransfected with the eukaryotic expression plasmid pTargeT-B7-H3 under a square pulse of 15 msec at a voltage of 160V and then cultured in an incubator at 37° C. and 5% CO₂. After 24 h, the cells were subjected to selection with culture medium containing 1000 μg/ml G418 (Gibco, #10131-027). After 16 days of transfection, the positive rate of the transfection pool was detected by flow cytometry. The cells in the pool with a higher positive rate were plated (according to a cell density of 1×10⁶ cells/ml, 100 μl/well, plated in a 96-well plate), and incubated with hBRCA84D antibody and Goat pAb to Hu IgG (PE) (Abcam, ab98596) antibody. Flow cytometer (ACEABIO, Novocyte 2060R) was used to read the mean value at a wavelength of 585 nm, and data analysis was performed using GraphPad. The positive cell lines were subcloned, and a CHO-K1 cell line was selected, which expressed B7-H3 at a high level and was named CHO-K1-B7-H3.

### (2) Expression of Tagged Antigen Protein and Positive Control Antibody

293E cells were inoculated into a 1 L cell culture flask with a density of 0.5×10⁶ cells/ml. Fresh and pre-warmed FreeStyle 293 expression medium was added to make the total volume of 250 mL. The cells were cultured in a humidified CO₂ incubator at 37° C. and 8% CO₂ overnight. 500 μl of 1 mg/ml PEI solution was added to 8.5 mL FreeStyle 293 expression medium and mixed well. 250 μg of plasmid to be transfected was added to 8.5 ml FreeStyle 293 expression medium and mixed well, wherein, the tagged antigen protein plasmids pHR-4Ig-B7-H3-hFc, pHR-4Ig-B7-H3-his, pHR-2Ig-B7-H3-hFc and pHR-2Ig-B7-H3-his were transfected respectively; the heavy chain plasmid pHR-hBRCA84D-8mIgG1 and the light chain plasmid pHR-hBRCA84D-hK of positive control antibody hBRCA84D were co-transfected; and the heavy chain plasmid pHR-hBRCA84D-mG2a and the light chain plasmid pHR-hBRCA84D-mK of murine-derived positive control antibody BRCA84D were co-transfected. The FreeStyle 293 expression medium containing PEI was added to the expression medium containing plasmid and mixed well, and then the mixture was added to the cells, and cultured in a humidified CO₂ incubator at 37° C. and 8% CO₂. The cells were fed on the 1st and 3rd day after cell transfection with 2.5 ml of 200 mM glutamine and 5 ml of 180 g/L glucose per flask. The cell culture supernatant was collected when the cell viability dropped to 65%˜75%. The cell culture was centrifuged at 1,500 rpm for 5 min to collect the supernatant, and then centrifuged at 8,000 rpm for 20 min to collect the supernatant.

### (3) Affinity Chromatography Purification

Different affinity chromatography columns were used to perform purification by using AKTA machine (GE, AKTA pure-150) according to the properties of the proteins (see Table 1 for affinity chromatography columns suitable for different proteins). The specific purification steps are as follows.

TABLE 1
Affinity chromatography columns suitable for different proteins

| Protein | Column | Brand | Model |
|---|---|---|---|
| Murine monoclonal | Protein G | Bestchrom | Ezfast |
| antibody (hybridoma), | prepacked | | Protein G 4FF |
| murine-derived | column | | |
| positive control | | | |
| antibody | | | |
| 4Ig-B7-H3-his/ | NI prepacked | GE | His Trap, HP 5 ml |
| 2Ig-B7-H3-his | column | | |
| 4Ig-B7-H3-hFc/ | Protein A | GE | Hi Trap, |
| 2Ig-B7-H3-hFc | prepacked | | MabselectSuRe |
| | column | | 5 ml |
| Chimeric antibody, | Protein A | GE | Mabselect |
| humanized antibody | self-packed | | LX 18 ml |
| | column | | |

Cleaning

The equipment and pipelines were cleaned with ultrapure water for 2 min with a flow rate of 10 mL/min, and then the chromatography system was cleaned with 0.1M NaOH.

Column Connection

The chromatography column was connected to the chromatography equipment and rinsed with ultrapure water for 5 min, and then the chromatography system was rinsed with 0.1M NaOH for 30 min with a retention time of 5 min.

Equilibration

Five CVs (column volume) of 20 mM PB+0.15M NaCl, pH 7.2 was used to equilibrate the column.

Sample Loading

The supernatant from cell expression was loaded to the column with a retention time of 5 min.

Post-Equilibration

Five CVs 20 mM PB+0.15M NaCl, pH 7.2 was used to equilibrate.

Elution

Elution was performed with 50 mM acetic acid (pH=3.4), for a retention time of 5 min. Collection started when UV280 reached about 50 mAu, and stopped when UV280 dropped to about 50 mAu. The sample was adjusted to the pH of 7.0 with 1M Tris-HCl (pH 9.0).

Re-Equilibration

Three CVs of 20 mM PB+0.15M NaCl, pH 7.2, was used to equilibrate with a retention time of 5 min.

On-Column Cleaning

Cleaning was performed with 0.1M NaOH for 30 min with a retention time of 5 min.

Cleaning and Preservation

Cleaning was performed with purified water for 10 minutes, and then 2 CVs of 20% ethanol.

### Example 2 Preparation of Monoclonal Antibodies

### 1. Preparation of Hybridomas

### (1) Animal Immunization

The experimental animals were immunized with different tagged 4Ig-B7-H3 antigen and 2Ig-B7-H3 antigen proteins with an adjuvant, and the experimental animals included SJL mice, Balb/c mice, and SD rats. The animals were immunized with 50 μg of antigen per animal for the first shoot, and subsequently 25 μg of antigen per animal was used for immunization. The immune adjuvant used in the experiments may be Freund's adjuvant (Sigma) or Quick Antibody-Mouse5W (Beijing Biodragon immunotech. Co., Ltd.). Freund's adjuvant was used to emulsify antigen and different tagged 4Ig-B7-H3 or 2Ig-B7-H3 antigen protein samples were added dropwise to the adjuvant solution with vortex to mix thoroughly. The dosages of the adjuvant were referred to the instructions. After the mixture was mixed well to form a water-in-oil emulsion, mice were immunized. Quick Antibody-Mouse5W was used as an adjuvant and different tagged 4Ig-B7-H3 or 2Ig-B7-H3 antigen protein samples were mixed with Quick Antibody-Mouse5W at a volume ratio of 1:1. After the mixture was mixed well, SD rats were immunized by intramuscular injection.

The stable transgenic cell line CHO-K1-B7-H3 expressing B7-H3 was used to immunize SD rats and Balb/c mice to produce anti-B7-H3 antibodies. The CHO-K1-B7-H3 cells obtained in Example 1 were treated with trypsin and then centrifuged at 1,000 rpm for 5 min. The cell supernatant was discarded and the cell pellet was resuspended in PBS. Part of the cells was taken out and counted with a cell counter, and the remaining cells were centrifuged at 1,000 rpm for 5 min. The supernatant was discarded and the cell pellet was resuspended in PBS. An appropriate amount of PBS was added to obtain a cell suspension of 1×10⁸ cells/ml. Each mouse in the experimental group was immunized with 1×10⁷ cells, and each SD rat in the experimental group was immunized with 2×10⁷ cells.

The immunization protocol is shown in Table 2.

TABLE 2
Animal immunization protocol

| | | | | Immunization |
|---|---|---|---|---|
| Group | Antigen | Animal | Adjuvant | Route |
| 1 | PBS | SJL/Balb/c/SD rat | None | s.c./i.m. |
| 2 | 4Ig-B7-H3-his/4Ig-B7-H3-hFc | SJL/Balb/c | Freund's adjuvant | s.c./i.m. |
| 3 | 2Ig-B7-H3-his/2Ig-B7-H3-hFc | Balb/c | Freund's adjuvant | s.c./i.m. |
| 4 | 4Ig-B7-H3-his/4Ig-B7-H3-hFc | SD rat | Quick | i.m. |
| | | | Antibody-Mouse5W | |
| 5 | CHO-K1-B7-H3 | Balb/c/SD rat | None | i.p. |

* i.m.: intramuscular injection; s.c.: subcutaneous injection; i.p.: intraperitoneal injection.

### (2) Hybridoma Fusion

Acquisition and preparation of spleen cells. The mice/rats after booster immunization were sacrificed and soaked in 75% alcohol. The spleen was dissected out, ground with a grinding rod, and filtered through a cell strainer to prepare a single cell suspension. The spleen cell suspension was centrifuged at 2,000 rpm for 5 min, and the supernatant was discarded. 2 mL red blood cell lysate was added to lyse red blood cells at room temperature for 2 min and PBS was added to reach 20 mL. After centrifugation at 1,500 rpm for 7 min, the supernatant was discarded. Viable cells were counted after resuspension. The Sp2/0 cells in the culture flask were collected and after centrifuged at 1,000 rpm for 5 min, the supernatant was discarded. Viable cells were counted after resuspension. The spleen cells were mixed with Sp2/0 cells at a ratio of 1:1 and subjected to centrifugation at 1,500 rpm for 7 min, the supernatant was discarded. The cells were resuspended in 20 mL electroporation buffer. After centrifugation at 1,500 rpm for 7 min, the supernatant was discarded and the step was repeated once. The cells were resuspended with an appropriate amount of electroporation buffer to ensure the cell concentration of about 2×10⁷ cells/mL. The cell suspension was added to a 9 mL electroporation tank for fusion. After fusion, the cell suspension was transferred to 15 mL RPMI 1640 complete medium containing 20% FBS and then left at room temperature for 20 min. The fused cells were resuspended with RPMI 1640 medium containing 1×HAT, 1×BIOMYC3, and 20% FBS. The cell suspension was added to several 96-well cell culture plates at 100 μl/well to ensure that the cell volume per well was about 4×10⁴ cells/well, and the plates was placed in a 37° C. cell incubator. After 5 days, additional 100 μL of RPMI 1640 complete medium containing 20% FBS, 1×HAT, and 1×BIOMYC-3 was added to each well.

### (3) Screening of Hybridoma and Subclones

After one week of fusion, the cell culture supernatants from hybridoma parent clones culture were collected and used for screening hybridoma parent clones binding to 4Ig-B7-H3-his protein and cynomolgus monkey B7-H3 protein by ELISA. The parent clones that can bind to CHO-K1-B7-H3 stable transgenic cell line were further screened by flow cytometry. The positive parent clones were expanded in 24-well plates, and the culture supernatants of the parent clones that had been cultured for 2 days were selected and co-cultured with T cells to detect the release of IFN-γ in the culture supernatant. The specific experimental operations are as follows:

1. On Day 1, T25 cell culture flasks were coated with 1 μg/ml anti-CD3e (R&D) and incubated overnight at 4° C. Liquid was discarded before the experiment, and the flasks were washed three times with PBS beforelater use.

2. On Day 2, CD8+ T cells (obtained by inducive expansion with CD3 and CD28, cryopreserved in 90% FBS+10% DMSO) were thawed, resuspended in culture medium with IMDM+10% FBS+free 100 ng/ml SEB+4 ng/ml IL-2+100× double antibiotics, and cultured in coated T25 in step 1 at a culture density of 2-3×10⁶ cells/ml for 48 h.

3. On Day 3, 96-well plates were coated with 1 μg/ml anti-CD3e and 10 μg/ml 4Ig-B7-H3-his, and divided into the coating blank group, anti-CD3e group and anti-CD3e+B7-H3-4Ig-His group. Plates were incubated overnight at 4° C.

4. On Day 4, the 96-well plates in the previous step was added with culture supernatant from hybridoma at 100 μl/well, with 100 μl of hybridoma medium added to control well, and incubate at 37° C. for 30 min.

5. CD8+T cells that had been stimulated for 2 days were collected and adjusted to a cell density of 1×10⁶ cells/ml, added to the 96-well plates at 100 μl/well, and cultured for 48 h.

6. On Day 6, IFN-γ release in the supernatant was tested according to R&D's duset (DY285B) detection kit to detect IFN-γ in the supernatant.

The parent clones with binding ability were screened by ELISA and FACS, and the release level of cytokine IFN-γ induced by the supernatant from the parent clones was further evaluated to screened out positive parent clones that could promote the release of IFN-γ. The positive parent clones were subcloned by the limiting dilution method. After one week of culture, the binding activity of the supernatant from the subclone to 4Ig-B7-H3 was detected by ELISA, and the monoclonal cell lines secreting anti-B7-H3 antibodies were then obtained.

### 2. Identification of Antibody Subtypes

The antibody subtypes were identified according to the instructions of the mouse antibody subtype identification kit “SBA Clonotyping Systerm-C57BL/6-HRP” (SouthernBiotech, Cat. No. 5300-05B). The results are shown in Table 3.

TABLE 3
Identification results of antibody subtypes

| Antibody | Antibody subtype |
|---|---|
| SHS007-17 | IgG/k |
| SHS007-39 | IgG/k |
| SHS007-51 | IgG/k |
| SHS007-65 | IgG/k |

### 3. Preparation of Monoclonal Antibodies

According to the activity analysis results of the supernatants from cell culture, the parent clones of monoclonal antibodies 17, 39, 51, and 65 were identified and expanded. The culture medium was 1640 medium containing 10% fetal bovine serum, 1× NAEE, 1× sodium pyruvate, and 1% penicillin-streptomycin double antibiotics. When the cell confluence was >80%, the cells were subcultured and expanded. 50 ml of the supernatant was collected and the antibody was purified. The obtained antibody was subjected to SDS-PAGE gel electrophoresis and showed a good purity.

### 4. Sequencing of Monoclonal Antibodies

The subcloned positive hybridomas were expanded, and an appropriate amount of cells was used for total RNA extraction according to the instructions of RNeasy Plus Mini Kit (Qiagen, 74134). The first strand of cDNA was synthesized using Prime Script 1st strand cDNA Synthesis Kit (Takara, 6110A).

Specific primers were designed according to the variable region of the mouse antibody subtype, and 5′ end of the primers contained the homologous arm sequence for homologous recombination with the eukaryotic expression vector. PCR amplification for the variable region of antibodies was performed using cDNA as a template to obtain the gene fragments of the light chain variable region and heavy chain variable region of the mouse antibody respectively. The design of primers refers to references: Anke Krebber, Susanne Bornhauser, Jorg Burmester et al. Reliable cloning of functional antibody variable domains from hybridomas and spleen cell repertoires employing a reengineered phage display system. Journal of Immunological Methods, 1997, 201: 35-55; 2. Simon KorenMihaKosmačAnjaColjaVenturinietal. Antibody variable-region sequencing as a method for hybridoma cell-line authentication, 2008, 78: 1071-1078. DNA sequencing was performed and the results are shown in Table 4.

TABLE 4
Sequence table of anti-B7-H3 murine-derived monoclonal antibody

| | Amino acid sequence of the | Amino acid sequence of the |
|---|---|---|
| Antibody | heavy chain variable region | light chain variable region |
| SHS007-17 | SEQ ID NO: 26 | SEQ ID NO: 27 |
| SHS007-39 | SEQ ID NO: 28 | SEQ ID NO: 29 |
| SHS007-51 | SEQ ID NO: 30 | SEQ ID NO: 31 |
| SHS007-59 | SEQ ID NO: 33 | SEQ ID NO: 34 |
| SHS007-65 | SEQ ID NO: 35 | SEQ ID NO: 36 |
| SHS007-72 | SEQ ID NO: 37 | SEQ ID NO: 38 |
| SHS007-90 | SEQ ID NO: 39 | SEQ ID NO: 40 |
| SHS007-95 | SEQ ID NO: 41 | SEQ ID NO: 42 |
| SHS007-108 | SEQ ID NO: 43 | SEQ ID NO: 44 |

### Example 3 Construction of Chimeric Antibodies

The gene fragments of heavy chain variable region of mouse antibodies obtained by sequencing as shown in Table 4 were cloned into an eukaryotic expression plasmid (pHR-8mIgG1) containing the heavy chain constant region with specific mutation sites (K214R, L235V, F243L, R292P, Y300L, D356E, L358M and P396L). The gene fragments of light chain variable region of mouse antibodies obtained by sequencing as shown in Table 4 were cloned into an eukaryotic expression plasmid (pHR-hK) containing human light chain constant region. The constructed plasmids expressing the heavy chain or light chain were transformed into Escherichia coli DH5a competent cells with the linearized eukaryotic expression plasmid containing the light chain constant region or the heavy chain constant region of the human antibody. The mixture was spread evenly on the surface of the agar plates containing the corresponding antibiotics. The agar plates were incubated in a 37° C. constant temperature incubator overnight, and then several single colonies were picked out for DNA sequencing. The positive clones with correct sequences were inoculated in 2×YT liquid medium containing the corresponding antibiotics and cultured at 37° C. for more than 12 hours with shaking. The bacterial cells were collected for plasmid extraction to obtain the plasmids expressing the light chain and the heavy chain of the chimeric antibody. The concentration and purity of the plasmids were detected by a nucleic acid quantitative analyzer.

The plasmids encoding chimeric antibody's heavy and light chains were co-transfected into HEK293E cells. The chimeric antibodies were expressed, purified and named SHS007-17-CHI, SHS007-39-CHI, SHS007-51-CHI and SHS007-65-CHI. The purity, activity, and affinity were then tested and analyzed.

The sequences of the chimeric antibodies SHS007-17-CHI, SHS007-39-CHI, SHS007-51-CHI and SHS007-65-CHI were subjected to gene mutation by site-directed mutagenesis to screen for better antibodies. G at position 34 of SHS007-51-CHI light chain CDR was mutated to K, and the obtained antibody had improved stability, and was marked as SHS007-51-G34K-CHI. The sequencing results of the chimeric antibodies are shown in Table 5.

TABLE 5
Sequences of anti-B7-H3 chimeric antibodies

| | Amino acid | Amino acid |
|---|---|---|
| | sequence of the | sequence of the |
| | heavy chain | light chain |
| Chimeric antibody | variable region | variable region |
| SHS007-17-CHI | SEQ ID NO: 26 | SEQ ID NO: 27 |
| SHS007-39-CHI | SEQ ID NO: 28 | SEQ ID NO: 29 |
| SHS007-51-CHI | SEQ ID NO: 30 | SEQ ID NO: 31 |
| SHS007-51-G34K-CHI | SEQ ID NO: 30 | SEQ ID NO: 32 |
| SHS007-65-CHI | SEQ ID NO: 35 | SEQ ID NO: 36 |

### Example 4 Construction and Production of Humanized Antibodies

According to the analysis of immune activity and killing activity of chimeric antibodies, SHS007-17-CHI, SHS007-39-CHI, SHS007-51-G34K-CHI and SHS007-65-CHI were modified to humanized antibodies.

To construct the humanized antibodies, the variable regions of SHS007-17-CHI, SHS007-39-CHI, SHS007-51-G34K-CHI and SHS007-65-CHI antibodies were compared with the mouse antibody sequences in the ImMunoGeneTics (IMGT) to determine their murine-derived germlines. After homology comparison, it was found that the FR region sequences of the heavy chain variable region of SHS007-17-CHI, SHS007-39-CHI, SHS007-51-G34K-CHI and SHS007-65-CHI antibodies were the most similar to the germline gene of the mouse antibody IGHV1-69*02, IGHV1-2*02, IGHV3-21*01 and IGHV3-48*01 respectively; the FR sequences of the light chain variable region of the antibodies were the most similar to the mouse antibody IGKV2-28*01, IGKV3-11*01, IGKV2-30*02 and IGKV3-11*01 respectively. With the framework region sequence FR1-FR3 of SHS007-17-CHI, SHS007-39-CHI, SHS007-51-G34K-CHI and SHS007-65-CHI antibodies as templates, full human framework regions with similar 3D structure but low immunogenicity were screened in the human framework region library to replace FR1-FR3 sequence of SHS007-17-CHI, SHS007-39-CHI, SHS007-51-G34K-CHI and SHS007-65-CHI. The full-length sequences of the heavy/light chain were 3D modeled and compared structurally with the heavy/light chain sequences of the original antibodies. Considering the antigenicity and 3D structural similarity, 4 humanized heavy chain variable regions (see SEQ ID NOs: 45, 46, 47 and 48) and 3 humanized light chain variable regions (see SEQ ID NOs: 49, 50 and 51) of SHS007-17-CHI, 6 humanized heavy chain variable regions (see SEQ ID NOs: 52, 53, 54, 55, 56 and 57) and 5 humanized light chain variable regions (see SEQ ID NOs: 58, 59, 60, 61 and 62) of SHS007-39-CHI, 2 humanized heavy chain variable regions (see SEQ ID NOs: 63 and 64) and 6 humanized light chain variable regions (see SEQ ID NOs: 65, 66, 67, 68, 69 and 70) of SHS007-51-G34K-2, and 3 humanized heavy chain variable regions (see SEQ ID NOs: 71, 72 and 73) and 6 humanized light chain variable regions (see SEQ ID NOs: 74, 75, 76, 77, 78 and 79) of SHS007-65-CHI were ultimately selected for further optimization. The sequences of non-CDR regions of the humanized antibodies of SHS007-17-CHI, SHS007-39-CHI, SHS007-51-G34K-CHI and SHS007-65-CHI all realized more than 95% humanization.

The amino acid sequences of the light chain and heavy chain variable region of humanized antibody obtained above were reversely transcribed into their corresponding nucleotide sequences, and oligonucleotide fragments containing complementary sequences between adjacent fragments were generated. The oligonucleotide fragments were annealed and assembled by Overlap PCR. Then nucleotide fragments of the entire light chain and heavy chain variable regions were amplified using specific primers (5′ end contained the homologous arm sequence for homologous recombination with the eukaryotic expression vector). The purified nucleotide fragments of the light chain variable region were co-transformed into Escherichia coli DH5α competent cells with the linearized eukaryotic expression plasmid (pHR-hK) containing light chain constant region of human K. The purified nucleotide fragments of the heavy chain variable region were co-transformed into Escherichia coli DH5a competent cells with the eukaryotic expression plasmid (pHR-IgG1) containing heavy chain constant region of human IgG1. The competent cells transformed with the plasmids were spread evenly on the surface of the agar plates containing the corresponding antibiotics. The agar plates were incubated in a 37° C. constant temperature incubator overnight, and then several single colonies were picked out for DNA sequencing.

The positive clones with correct sequences were inoculated in 2×YT liquid medium containing the corresponding antibiotics and cultured at 37° C. with shaking for more than 12 hours. The bacterial cells were collected for plasmid extraction to obtain the expression plasmids for the light chain and the heavy chain of humanized antibodies. The concentration and purity of the plasmids were detected by a nucleic acid quantitative analyzer.

The plasmids were transfected into HEK293E cells, and a large number of antibodies were expressed and purified. The purity, activity and affinity were tested and analyzed.

The humanized antibodies with good purity, activity and affinity were selected, and named as hu17-00, hu39-01, hu65-13 and hu51-G34K-12. The sequences are shown in Table 6.

TABLE 6
Sequence table of anti-B7-H3 humanized antibodies

| | Amino acid sequence | | Nucleotide sequence | |
|---|---|---|---|---|
| | Heavy chain | Light chain | Heavy chain | Light chain |
| Humanized | variable | variable | variable | variable |
| antibody | region | region | region | region |
| hu17-00 | SEQ ID | SEQ ID | SEQ ID | SEQ ID |
| | NO: 45 | NO: 49 | NO: 80 | NO: 81 |
| hu39-01 | SEQ ID | SEQ ID | SEQ ID | SEQ ID |
| | NO: 52 | NO: 59 | NO: 82 | NO: 83 |
| hu51- | SEQ ID | SEQ ID | SEQ ID | SEQ ID |
| G34K-12 | NO: 64 | NO: 67 | NO: 84 | NO: 85 |
| hu65-13 | SEQ ID | SEQ ID | SEQ ID | SEQ ID |
| | NO: 72 | NO: 77 | NO: 86 | NO: 87 |

### Example 5 Using ELISA to Measure Binding Activity of the Antibodies to Human B7-H3

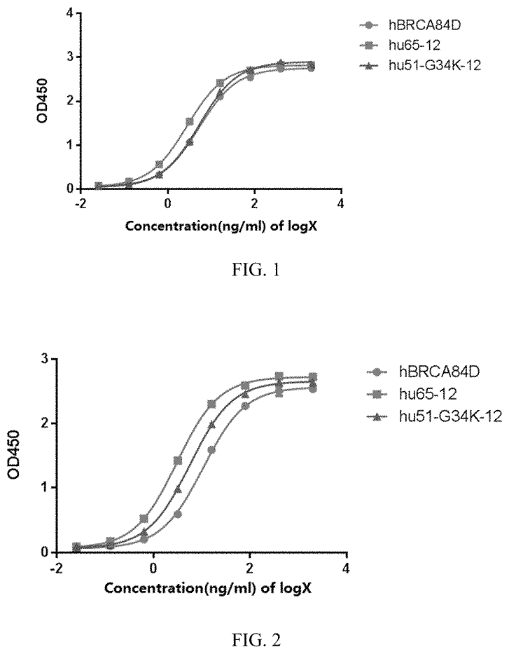

The binding activity of antibodies was analyzed by ELISA. 96-well ELISA plates were coated with human 4Ig-B7-H3-His protein or 2Ig-B7-H3-His protein (1 μg/well, prepared in Examples 1 and 2) and incubated overnight at 4° C. The plates were washed 3 times with 1×PBST and then blocked with 5% skim milk at 37° C. for 2 h. After washing 3 times with 1×PBST, the anti-B7-H3 antibody of the present disclosure was used as the primary antibody and added to the ELISA plates in 5-fold gradient dilution starting from 2 μg/mL with a total of 8 concentrations: 2,000 ng/mL, 400 ng/mL, 80 ng/mL, 16 ng/mL, 3.2 ng/mL, 0.64 ng/mL, 0.128 ng/mL and 0.0256 ng/mL respectively. The plates were incubated at 37° C. for 2 h with hBRCA84D as the positive control antibody. After washing 5 times with 1×PBST, Anti-Human IgG HRP (Jackson, 109-035-003, 1:5000) was used as the secondary antibody and incubated at 37° C. for 1 h. After washing 5 times with 1×PBST, color developing solution TMB was added to the plate, and microplate reader (Thermo, Multiskan FC) was used to read OD450 value after termination of the reaction. EC₅₀ was generated by GraphPad. The result is shown in FIG. 1 and FIG. 2.

The experimental results show that the humanized anti-B7-H3 antibodies hu51-G34K-12 and hu65-13 of the present disclosure had the ability to bind to human 4Ig-B7-H3 and 2Ig-B7-H3, and the binding ability was comparable to that of the positive control antibody hBRCA84D.

### Example 6 Using ELISA to Measure Binding Activity of the Antibodies to Cynomolgus Monkey B7-H3

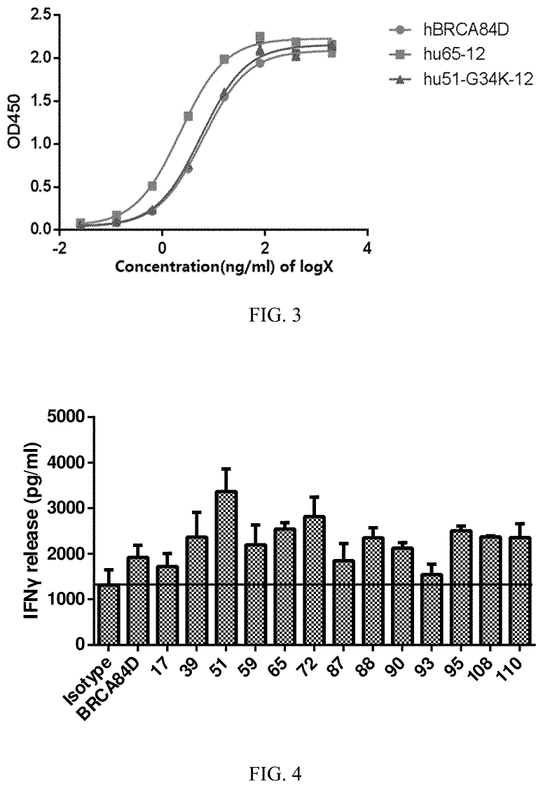

The binding activity of antibodies was analyzed by protein based ELISA. Cynomolgus B7-H3-His (0.5 μg/well, Sino Biological, Cat. No. 90806-C08H) was coated on 96-well ELISA plates. The anti-B7-H3 antibodies of the present disclosure was used as the primary antibody and added to the ELISA plates in 5-fold gradient dilution starting from 2 μg/mL with a total of 8 concentrations: 2,000 ng/mL, 400 ng/mL, 80 ng/mL, 16 ng/mL, 3.2 ng/mL, 0.64 ng/mL, 0.128 ng/mL, and 0.0256 ng/mL respectively. The plates were incubated at 37° C. for 2 h with AB06.12-4P as the positive control antibody. Anti-Human IgG HRP (Jackson, 109-035-003, 1:10,000) was used as the secondary antibody. Color developing solution TMB (3,3′,5,5′-tetramethylbenzidine) was added to the plate, and microplate reader (Thermo, Multiskan FC) was used to read OD450 value after termination of the reaction. EC₅₀ was generated by GraphPad. The result is shown in FIG. 3.

The experimental results show that the humanized anti-B7-H3 antibodies hu51-G34K-12 and hu65-13 of the present disclosure had the ability to bind to cynomolgus B7-H3, and the binding ability was comparable to that of the positive control antibody hBRCA84D.

### Example 7 Using FACS to Measure Binding Activity of the Antibodies to B7-H3 on Surface of Tumor Cell

The binding activity of antibodies to B7-H3 on the surface of tumor cells was analyzed by FACS. Mean fluorescence intensity (MFI) and EC₅₀ detected by FACS indicate the binding activity of the antibody to cells. After 786-0 and MDA-MB-231 cells were digested, they were resuspended in 2% FBS-PBS solution and counted. After raji-B7-H3 cells were collected, they were resuspended in 2% FBS-PBS and counted. The above cells were plated in a cell plate with 1×10⁵ cells per well. The anti-B7-H3 antibody of the present disclosure was used as the primary antibody and added to cell plates in gradient dilution starting from 20 μg/mL with a total of 8 concentrations: 20,000 ng/mL, 10,000 ng/mL, 2,000 ng/mL, 400 ng/mL, 80 ng/mL, 16 ng/mL, 3.2 ng/mL, and 0.64 ng/mL respectively. The plates were incubated at 4° C. for 1 h with hBRCA84D as the positive control antibody. PE-Anti-Human IgG (Biolegend, Cat. No. 409303, 1.25 μl/well) was used as the secondary antibody, washed, and detected for the fluorescence intensity generated by the binding of the antibody to the cell surface by a flow cytometer. EC₅₀ was calculated using GraphPad and the results are shown in Tables 7a and 7b.

TABLE 7a
Assay for binding to B7-H3 on surface of tumor cell

| Tumor | Mean fluorescence intensity (MFI) | | |
|---|---|---|---|
| cell type | hBRCA84D | hu65-13 | hu51-G34K-12 |
| 786-0 | 2.33E+05 | 5.66E+05 | 5.84E+05 |
| MDA-MB-231 | 3.58E+04 | 1.51E+05 | 1.56E+05 |
| Raji-B7-H3 | 2.33E+05 | 5.66E+05 | 5.84E+05 |

TABLE 7b
Assay for binding to B7-H3 on surface of tumor cell

| Tumor | EC₅₀ (μg/ml) | | |
|---|---|---|---|
| cell type | hBRCA84D | hu65-13 | hu51-G34K-12 |
| 786-0 | 0.2256 | 0.2658 | 0.1199 |
| MDA-MB-231 | 0.3844 | 0.1403 | 0.0501 |
| Raji-B7-H3 | 0.2256 | 0.2658 | 0.1199 |

The experimental results show that the humanized anti-B7-H3 antibodies, hu51-G34K-12 and hu65-13, can bind to B7-H3 on the surface of tumor cells, and the binding density to tumor cells was significantly higher than that of the control antibody hBRCA84D. Both EC₅₀ and Emax of the binding were significantly better than that of the control antibody hBRCA84D.

### Example 8 Using Biacore to Measure Binding Activity of the Antibodies

The humanized anti-B7-H3 antibodies prepared in Example 4 were subjected to affinity assay by Biacore. The antigen 4Ig-B7-H3-His was first coupled to the surface of a CM5 chip (GE, Cat. No. BR100012), and the active groups of the uncoupled protein were blocked with ethanolamine. After the baseline was stable, the hu39-01, hu65-13, and hBRCA84D antibodies were respectively diluted in equal proportions. The diluted antibodies of each concentration were placed on the sample injection tray of the instrument in sequence, and the program was set to inject samples respectively. The affinities between hu51-G34K-12, hu65-13, hBRCA84D antibodies and 4Ig-B7-H3-His antigen were determined according to the operating procedures of Biacore X100. The specific parameters and experimental results are shown in Table 8.

TABLE 8
Affinity of antibody to human B7-H3 protein

| Antibody | KD (M) | kon (1/Ms) | kdis (1/s) |
|---|---|---|---|
| hu51-G34K-12 | 4.90E−10 | 7.85E+05 | 3.85E−04 |
| hu65-13 | 1.98E−10 | 9.54E+04 | 1.89E−05 |
| hBRCA84D | 4.55E−10 | 2.43E+05 | 1.11E−04 |

The experimental results show that hu51-G34K-12 and hu65-13 bound to human 4Ig-B7-H3 protein with good affinity, and the affinity value was comparable to that of the positive control antibody hBRCA84D.

### Example 9 Detection of Blocking Activity of B7-H3 Murine Monoclonal Antibody and Humanized Antibody by IFN-γ Release

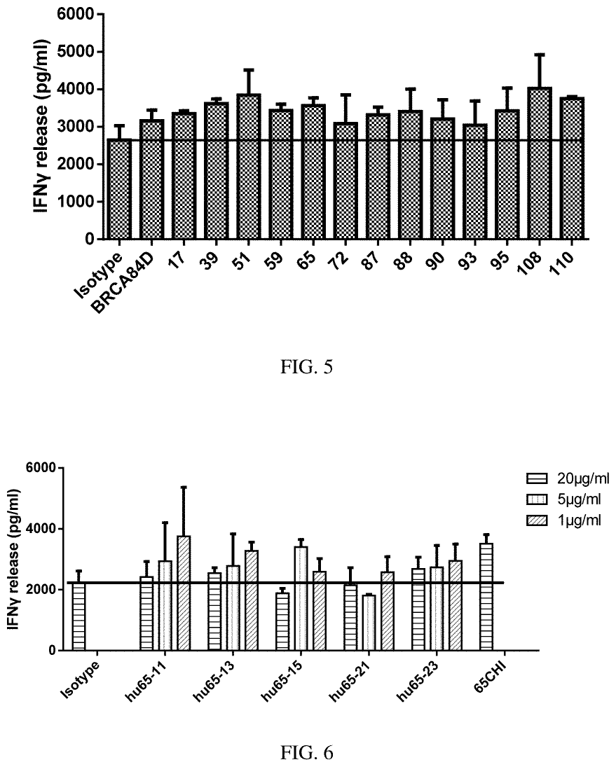

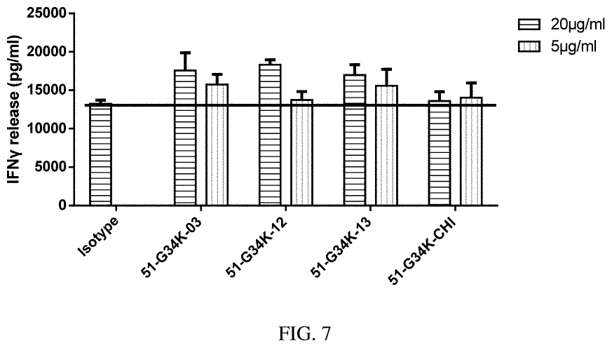

Human peripheral blood mononuclear cells (PBMCs) were isolated by Ficoll (GE Healthcare, Cat. No. 10268731). The isolated PBMCs were resuspended in IMDM (Life technologies, 12440053) complete medium containing 10% FBS and adjusted to a density of 1*10⁶ cells/mL. PB MC cells were added to a 96-well plate at 100 μL/well and allowed to stand for 30 min. The antibody to be detected and the isotype antibody were prepared in the same medium to a final concentration of 20 μg/ml, added to the 96-well plate, and incubated in a constant temperature incubator at 37° C. for 1 h. SEB (Toxin techonology, BT202) solution with a final concentration of 100 ng/ml was prepared with the culture medium and added to the corresponding wells of the 96-well plate. After co-culture in the 96-well plate in the constant temperature incubator for 72 h, the concentration of IFN-γ in the culture supernatant was detected according to the instructions of DuoSet detection kit (R&D, DY285B). FIG. 4 and FIG. 5 show that anti-human B7-H3 murine monoclonal antibodies SHS007-39, 51, 59, 65, 72, etc., in the PBMC-SEB system derived from donor B and donor C, increased the release of cytokine IFN-γ in the system. FIG. 6 and FIG. 7 show that anti-human B7-H3 humanized antibodies hu51 and hu65 increased the release of IFN-γ in the PBMC-SEB experimental system.

### Example 10 Antibody-Dependent Cell-Mediated Cytotoxicity (ADCC) Assay (LDH Method)

Human renal carcinoma cells 786-0 have a high expression level of human B7-H3. 786-0 cells were plated in 96-well plates at 20,000 cells per well. RPMI-1640 medium containing 2% FBS was prepared with heat-inactivated fetal bovine serum (FBS, Gibco, Cat. No. 10091-148). SHS007-17CHI, SHS007-39CHI, SHS007-51CHI, SHS007-65CHI, hBRCA84D antibodies were diluted in 10-fold gradient dilution in the above medium, and incubated in a constant temperature incubator at 37° C. for 1 h. According to the instructions of Ficoll (GE Healthcare, Cat. No. 10268731) kit, human peripheral blood PBMCs were isolated as effector cells, and the PBMC cells and the above antibody solution were added to the 96-well cell plates plated with 786-0 cells. The cell plates were incubated in a 37° C. incubator for 4 h, and detected for cytotoxicity according to the instructions of Cytotoxicity LDH Assay Kit-WST® (Dojindo, CK12). The fold reduction of the killing EC₅₀ of antibodies of the present disclosure (i.e., the ratio of the EC₅₀ value of the positive control hBRCA84D to the EC₅₀ value of the antibody of the present disclosure) was calculated. The experimental results are shown in Table 9.

TABLE 9
ADCC activity of chimeric antibodies

| Antibody | EC₅₀ Ratio |
|---|---|
| hBRCA84D | 1 |
| SHS007-17CHI | 20.1 |
| SHS007-39-CHI | 211.5 |
| SHS007-51-CHI | 6.6 |
| SHS007-59-CHI | 4.4 |
| SHS007-65-CHI | 18.6 |
| SHS007-72-CHI | 7.1 |
| SHS007-95-CHI | 11.4 |

The experimental results show that, compared with the positive control hBRCA84D antibody, the antibodies of the present disclosure had a better EC₅₀ value and greatly improved the killing activity of ADCC.

### Example 11 Anti-Tumor Test in Mouse Xenograft Model from Human Tumor Cells

### 1. Experimental Materials

### (1) Experimental Cells and Animals

Human malignant glioma U87 cells were purchased from the American Type Culture Collection (ATCC).

Human renal carcinoma cells 786-0 cells were purchased from the American Type Culture Collection (ATCC).

Human triple-negative breast cancer MDA-MB-231 cells were purchased from the American Type Culture Collection (ATCC).

SCID-Beige mice, female, 6-8 weeks old, weighing 18-20 grams, were purchased from Shanghai Lingchang Laboratory Animal Co., Ltd.

### (2) Test Samples and Controls

Negative control isotype IgG1 (Cat. No. C0001-4) was purchased from Crown Bioscience Co., Ltd. In the mouse xenograft model, the effect of hBRCA84D-IgG1 was much better than that of hBRCA84D, so hBRCA84D-IgG1 was used as a positive control. hBRCA84D-IgG1 was a human IgG1 monoclonal antibody constructed using the variable region sequence of the reference antibody hBRCA84D, of which the constant region was the same as the human IgG1 chimeric antibody of the present disclosure.

Before the test, the anti-B7-H3 chimeric antibodies of the present disclosure (SHS007-39-IgG1 and SHS007-65-IgG1) were prepared in PBS at 1 mg/mL, and isotype IgG1 and hBRCA84D-IgG1 were prepared at 1 mg/mL.

### 2. Experimental Method

### (1) U87 Xenograft Model

U87 cells were inoculated on the right rear of the mice at 5×10⁶ cells/mouse. Grouping was performed when the tumors were at an average volume of 140 mm³. Mice were administered twice a week through tail vein, and the diameter of the tumor was measured with a vernier caliper twice a week to calculate the tumor volume according to V=0.5a×b², where a and b represented the long and short diameters of the tumor, respectively. The tumor volume was measured and recorded for 24 days of continuous administration, and the tumor growth curve was plotted with GraphPad Prism. The results are shown in Table 10.

TABLE 10
Results of anti-tumor test in human
malignant glioma U87 xenograft model

| Antibody | Dose (mg/kg) | T/C (%) |
|---|---|---|
| Isotype IgG1 | 10 | 100 |
| hBRCA84D-IgG1 | 10 | 56.9 |
| SHS007-39-CHI | 10 | 41.3 |

### (2) 786-0 Xenograft Model

786-0 cells were inoculated on the right rear of the mice at 1×10⁷ cells/mice. After 2 weeks of inoculation, the tumor mass formed by 786-0 cell inoculation was removed with sterile surgical instruments, and the tumor tissue in the vigorous growth stage was cut into about 30 mm³ in normal saline, and inoculated into the right back of mice under sterile conditions. Grouping was performed when the tumor grew to an average volume of 160 mm³. Mice were administered twice a week through tail vein, and the diameter of the tumor was measured with a vernier caliper twice a week to calculate the tumor volume according to V=0.5a×b², where a and b represented the long and short diameters of the tumor, respectively. The tumor volume was measured and recorded for 19 days of continuous administration, and the tumor growth curve was plotted with GraphPad Prism. The results are shown in Table 11.

TABLE 11
Results of anti-tumor test in human
renal carcinoma cell 786-0 xenograft model

| Antibody | Dose (mg/kg) | T/C (%) |
|---|---|---|
| Isotype IgG1 | 10 | 100 |
| hBRCA84D-IgG1 | 10 | 69.78 |
| SHS007-39-CHI | 10 | 63.93 |
| SHS007-65-CHI | 10 | 52.94 |

### (3) MDA-MB-231 Xenograft Model

MDA-MB-231 cells were inoculated on the right rear of the mice at 8×10⁶ cells/mice. After 2 weeks of inoculation, the tumor mass formed by MDA-MB-231 cell inoculation was removed with sterile surgical instruments, and the tumor tissue in the vigorous growth stage was cut into about 30 mm³ in normal saline, and inoculated into the right back of mice under sterile conditions. Grouping was performed when the tumor grew to an average volume of 150 mm³. Mice were administered twice a week through tail vein, and the diameter of the tumor was measured with a vernier caliper twice a week to calculate the tumor volume according to V=0.5a×b², where a and b represented the long and short diameters of the tumor, respectively. The tumor volume was measured and recorded for 21 days of continuous administration, and the tumor growth curve was plotted with GraphPad Prism. The results are shown in Table 12.

TABLE 12
Results of anti-tumor test in human
triple-negative breast cancer cell
MDA-MB-231 xenograft model

| Antibody | Dose (mg/kg) | T/C (%) |
|---|---|---|
| Isotype IgG1 | 10 | 100 |
| hBRCA84D-IgG1 | 10 | 86.33 |
| SHS007-39-CHI | 10 | 62.84 |
| SHS007-65-CHI | 10 | 54.26 |

The experimental results show that the antibodies of the present disclosure had the effect of inhibiting tumor growth in the SCID-Beige mouse xenograft model inoculated with human malignant glioma U87 cells, human renal carcinoma cells 786-0 cells, and human triple-negative breast cancer cells MDA-MB-231. SHS007-39-CHI and SHS007-65-IgG1 had better effect on inhibiting tumor than the control antibody hBRCA84D-IgG1. The anti-B7-H3 antibodies of the present disclosure had a more significant effect of inhibiting tumor growth.

### Example 12 Anti-Tumor Test in Mouse Xenograft Model from Human Tumor Cells

### 1. Experimental Materials

### (1) Experimental Cells and Animals

Human renal carcinoma cells 786-0 cells were purchased from the American Type Culture Collection (ATCC).

Human triple-negative breast cancer MDA-MB-231 cells were purchased from the American Type Culture Collection (ATCC).

SCID-Beige mice, female, 6-8 weeks old, weighing 18-20 grams, were purchased from Shanghai Lingchang Laboratory Animal Co., Ltd.

### (2) Test Samples and Controls

The control isotype IgG1 (Cat. No. C0001-4) was purchased from Crown Bioscience Co., Ltd. and used as a negative control. The control hBRCA84D-IgG1 was a human IgG1 monoclonal antibody constructed using the variable region sequence of the reference antibody hBRCA84D, of which the constant region was the same as the humanized chimeric antibody of the present disclosure, and used as a positive control.

Before the test, the humanized anti-B7-H3 antibodies of the present disclosure hu65-13 and hu51-G34K-12 were prepared in PBS at 1 mg/mL or 2 mg/mL, and isotype IgG1 and hBRCA84D-IgG1 were prepared at 1 mg/mL or 2 mg/mL.

### 2. Experimental Method

### (1) 786-0 Xenograft Model

786-0 cells were inoculated on the right rear of the mice at 1×10⁷ cells/mice. After 2 weeks of inoculation, the tumor mass formed by 786-0 cell inoculation was removed with sterile surgical instruments, and the tumor tissue in the vigorous growth stage was cut into about 15 mm³ in normal saline, and inoculated into the right back of mice under sterile conditions. Grouping was performed when the tumor grew to an average volume of 100 mm³. Mice were administered twice a week through tail vein until day 21, and then the frequency of administration was reduced to once a week. The diameter of the tumor was measured with a vernier caliper twice a week to calculate the tumor volume according to V=0.5a×b², where a and b represented the long and short diameters of the tumor, respectively. The tumor volume was measured and recorded for 36 days of continuous administration, and the tumor growth curve was plotted with GraphPad Prism. The results are shown in Table 13.

TABLE 13
Results of anti-tumor test in human renal
carcinoma cell 786-0 xenograft model

| Antibody | Dose (mg/kg) | T/C (%) |
|---|---|---|
| Isotype | 10 | 100 |
| hBRCA84D-IgG1 | 10 | 85.4 |
| hu51-G34K-12 | 10 | 52.0 |
| hu65-13 | 10 | 60.8 |

### (2) MDA-MB-231 Xenograft Model

Human tumor cell MDA-MB-231 cells were inoculated on the right rear of the mice at 8×10⁶ cells/mice. After 2 weeks of inoculation, the tumor mass formed by MDA-MB-231 cell inoculation was removed with sterile surgical instruments, and the tumor tissue in the vigorous growth stage was cut into about 30 mm³ in normal saline, and inoculated into the right back of mice under sterile conditions. Grouping was performed when the tumor grew to an average volume of 100 mm³. Mice were administered twice a week through tail vein, and the diameter of the tumor was measured with a vernier caliper twice a week to calculate the tumor volume according to V=0.5a×b², where a and b represented the long and short diameters of the tumor, respectively. The tumor volume was measured and recorded for 25 days of continuous administration, and the tumor growth curve was plotted with GraphPad Prism. The results are shown in Table 14.

TABLE 14
Results of anti-tumor test in human triple-negative
breast cancer cell MDA-MB-231 xenograft model

| Antibody | Dose (mg/kg) | T/C (%) |
|---|---|---|
| Isotype IgG1 | 10 | 100 |
| hBRCA84D-IgG1 | 20 | 59.38 |
| hu65-13 | 10 | 52.55 |

The experimental results show that the humanized antibodies of the present disclosure hu51-G34K-12 and hu65-13 had the effect of inhibiting tumor growth in the SCID-Beige mouse xenograft model inoculated with human renal carcinoma cells 786-0 cells and human triple-negative breast cancer cells MDA-MB-231. hu51-G34K-12 and hu65-13 had better effect on inhibiting tumor than the control antibody hBRCA84D-IgG1. The anti-B7-H3 antibodies of the present disclosure had a more significant effect of inhibiting tumor growth.

The above examples demonstrate that the anti-B7-H3 antibodies of the present disclosure have a significant anti-tumor effect and can significantly inhibit tumor growth, suggesting that the antibody can be used in the manufacture of anti-tumor drugs and has a good market prospect.

Although the present disclosure has been described above in detail, those skilled in the art should understand that various modifications and changes can be made to the present disclosure without departing from the spirit and scope of the present disclosure. The scope of the present disclosure should not be limited to the detailed description above, but should be attributable to the appended claims.

1. An anti-B7-H3 antibody or antigen-binding fragment thereof, comprising a heavy chain variable region and a light chain variable region, wherein
(1) the heavy chain variable region comprises HCDR1, HCDR2 and HCDR3, selected from the group consisting of:
(a4) amino acid sequences as set forth in SEQ ID NOs: 20, 21 and 22, and
(a5) CDRs having at least 85% sequence identity to the amino acid sequences set forth in (a4); and
(2) the light chain variable region comprises LCDR1, LCDR2 and LCDR3, selected from the group consisting of:
(a10) amino acid sequences as set forth in SEQ ID NOs: 23, 24 and 25, and
(a11) CDRs having at least 85% sequence identity to the amino acid sequences set forth in (a10).

2. The anti-B7-H3 antibody or antigen-binding fragment thereof according to claim 1, wherein
the heavy chain variable region comprises HCDR1, HCDR2, and HCDR3 set forth in SEQ ID NOs: 20, 21 and 22 respectively, and the light chain variable region comprises LCDR1, LCDR2 and LCDR3 set forth in SEQ ID NOs: 23, 24 and 25 respectively.

3. The anti-B7-H3 antibody or antigen-binding fragment thereof according to claim 1, wherein the antibody is a murine-derived monoclonal antibody, a human-murine chimeric antibody, or a humanized antibody.

4. The anti-B7-H3 antibody or antigen-binding fragment thereof according to claim 1, wherein
(1) the amino acid sequence of the heavy chain variable region is selected from the group consisting of:
(b1) amino acid sequences as set forth in SEQ ID NO: 35,
(b2) amino acid sequences derived from the amino acid sequences set forth in (b1) by substitution, deletion or addition of one or more amino acids and functionally identical or similar to the amino acid sequences set forth in (b 1), and
(b3) amino acid sequences having at least 80% sequence identity to the amino acid sequences set forth in (b1); and
(2) the amino acid sequence of the light chain variable region is selected from the group consisting of:
(b4) amino acid sequences as set forth in SEQ ID NO: 36,
(b5) amino acid sequences derived from the amino acid sequences set forth in (b4) by substitution, deletion or addition of one or more amino acids and functionally identical or similar to the amino acid sequences set forth in (b4), and
(b6) amino acid sequences having at least 80% sequence identity to the amino acid sequences set forth in (b4).

5. The anti-B7-H3 antibody or antigen-binding fragment thereof according to claim 4, wherein the antibody further comprises a heavy chain constant region of murine-derived IgG1, IgG2, or a variant thereof, and a light chain constant region of murine-derived kappa chain or a variant thereof.

6. The anti-B7-H3 antibody or antigen-binding fragment thereof according to claim 1, wherein
the amino acid sequence of the heavy chain variable region is SEQ ID NO: 35 and the amino acid sequence of the light chain variable region is SEQ ID NO: 36.

7. The anti-B7-H3 antibody or antigen-binding fragment thereof according to claim 1, wherein the anti-B7-H3 antibody is a humanized antibody, wherein
(1) the amino acid sequence of the heavy chain variable region is selected from the group consisting of:
(c1) amino acid sequences as set forth in SEQ ID NO: 72 and SEQ ID NO: 73,
(c2) amino acid sequences derived from the amino acid sequences set forth in (c1) by substitution, deletion or addition of one or more amino acids and functionally identical or similar to the amino acid sequences as set forth in (c1), and
(c3) amino acid sequences having at least 80% sequence identity to the amino acid sequences set forth in (c1); and
(2) the amino acid sequence of the light chain variable region is selected from the group consisting of:
(c4) amino acid sequences as set forth in SEQ ID NO: 74, SEQ ID NO: 75, SEQ ID NO: 76, SEQ ID NO: 77, SEQ ID NO: 78 and SEQ ID NO: 79,
(c5) amino acid sequences derived from the amino acid sequences set forth in (c4) by substitution, deletion or addition of one or more amino acids and functionally identical or similar to the amino acid sequences as set forth in (c4), and
(c6) amino acid sequences having at least 80% sequence identity to the amino acid sequences set forth in (c4).

8. The anti-B7-H3 antibody or antigen-binding fragment thereof according to claim 7, wherein
the amino acid sequence of the heavy chain variable region is selected from the group consisting of SEQ ID NO: 72, the amino acid sequence derived from SEQ ID NO: 72 by substitution, deletion or addition of one or more amino acids and functionally identical to SEQ ID NO: 72, or the amino acid sequence having at least 85% sequence identity to SEQ ID NO: 72; and the amino acid sequence of the light chain variable region is selected from the group consisting of SEQ ID NO: 77, the amino acid sequence derived from SEQ ID NO: 77 by substitution, deletion or addition of one or more amino acids and functionally identical to SEQ ID NO: 77, and the amino acid sequence having at least 85% sequence identity to SEQ ID NO: 77.

9. An isolated nucleic acid encoding the anti-B7-H3 antibody or antigen-binding fragment thereof according to claim 1.

10. The nucleic acid according to claim 9, wherein
(1) the nucleotide sequence encoding the amino acid sequence of the heavy chain variable region is SEQ ID NO: 86; and
(2) the nucleotide sequence encoding the amino acid sequence of the light chain variable region is SEQ ID NO: 87.

11. An expression vector comprising the nucleic acid according to claim 9.

12. A host cell, wherein the host cell is transformed with the expression vector according to claim 11, and wherein the host cell is selected from the group consisting of prokaryotic cells and eukaryotic cells.

13. A method for producing the anti-B7-H3 antibody or antigen-binding fragment thereof according to claim 1, comprising
expressing the antibody in a host cell, and
isolating the antibody from the host cell;
wherein the host cell is transformed with ab expression vector comprising the nucleic acid encoding the anti-B7-H3 antibody or antigen-binding fragment thereof.

14. A pharmaceutical composition comprising the anti-B7-H3 antibody or antigen-binding fragment thereof according to claim 1 and a pharmaceutically acceptable carrier.

15. A method for inhibiting B7-H3 activity comprising administering the anti-B7-H3 antibody or antigen-binding fragment thereof according to claim 1 to a subject in need thereof.

16. A method for treating a cancer comprising administering the anti-b7-h3 antibody or antigen-binding fragment thereof according to claim 1 to a subject in need thereof, wherein the cancer is selected from the group consisting of breast cancer, kidney cancer, lung cancer, gastric cancer, intestinal cancer, esophageal cancer, ovarian cancer, cervical cancer, bladder cancer, pancreatic cancer, glioma, leukemia, lymphoma, and melanoma.
